(19) United States
(12) Patent Application Publication
(10) Pub. No.: US 2022/0347257 A1
(43) Pub. Date: Nov. 3, 2022

(54) QUORUM-SENSING INHIBITORS AND/OR POSTBIOTIC METABOLITES AND RELATED METHODS

(71) Applicant: MICROSINTESIS INC., Charlottetown, Prince Edward Island (CA)

(72) Inventor: Monica Angela Cella, Cornwall, Prince Edward Island (CA)

(21) Appl. No.: 17/624,455

(22) PCT Filed: Jul. 2, 2020
(86) PCT No.: PCT/CA2020/050921

§ 371 Date: Jan. 3, 2022

Related U.S. Application Data
(60) Provisional application No. 62/869,681, filed on Jul. 2, 2019.

(51) Int. Cl. A61K 38/08; A61K 38/10; A61K 45/06; C12N 1/20

(52) U.S. Cl. CPC A61K 38/08; A61K 38/10; A61K 45/06; C12N 1/205; A61K 2035/115

(57) ABSTRACT

Described herein is a synergistic combination comprising a quorum-sensing inhibitor and/or postbiotic metabolite and an antibiotic. Typically, the postbiotic metabolite comprises at least one peptide. Related compositions, uses, and methods are also described, including methods for resensitizing resistant bacteria to an antibiotic, and methods of treating antibiotic-resistant infections, such as methicillin-resistant Staphylococcus aureus (MRSA).

### FIELD

The present invention relates to quorum-sensing inhibitors and/or postbiotic metabolites. More specifically, the present invention is, in aspects, concerned with quorum-sensing inhibitors and/or postbiotic metabolites as alternatives to antibiotics, combinations of quorum-sensing inhibitors and/or postbiotic metabolites with antibiotics, and related compositions and methods.

### BACKGROUND

A World Health Organization (WHO) report released April 2014 stated, “this serious threat is no longer a prediction for the future, it is happening right now in every region of the world and has the potential to affect anyone, of any age, in any country. Antibiotic resistance—when bacteria change so antibiotics no longer work in people who need them to treat infections—is now a major threat to public health.” The European Centre for Disease Prevention and Control calculated that in 2015 there were 671,689 infections in the EU and European Economic Area caused by antibiotic-resistant bacteria, resulting in 33,110 deaths. Most were acquired in healthcare settings.

The World Health Organization concluded that inappropriate use of antibiotics in animal husbandry is an underlying contributor to the emergence and spread of antibiotic-resistant germs, and that the use of antibiotics as growth promoters in animal feeds should be restricted. The World Organisation for Animal Health has added to the Terrestrial Animal Health Code a series of guidelines with recommendations to its members for the creation and harmonization of national antimicrobial resistance surveillance and monitoring programs, monitoring of the quantities of antibiotics used in animal husbandry, and recommendations to ensure the proper and prudent use of antibiotic substances. Another guideline is to implement methodologies that help to establish associated risk factors and assess the risk of antibiotic resistance.

Since the discovery of antibiotics, research and development (R&D) efforts have provided new drugs in time to treat bacteria that became resistant to older antibiotics, but in the 2000s there has been concern that development has slowed enough that seriously ill people may run out of treatment options. Another concern is that doctors may become reluctant to perform routine surgeries because of the increased risk of harmful infection.

International Patent Application Publication Nos. WO 2009/155711, WO 2015/021530, 2018/165764, and WO 2018/165765 describe certain postbiotic metabolites that are found in the cell-free spent medium (CFSM) of probiotic cultures. These postbiotic metabolites have been shown to be effective in treating a number of different infections, including bacterial and viral infections of many different origins and in different species.

Despite this, there remains a need for alternatives to conventional antibiotics and methods for treating infection.

### DESCRIPTION OF THE DRAWINGS

The present invention will be further understood from the following description with reference to the Figures, in which:

FIG. 1. (a,b) Heat plots of MIC percent growth inhibition of the two respective clinical MRSA strains by combination testing of the β-lactam antibiotic cefoxitin (0-100 μg/mL) and DSM13241 bioactive metabolites (5, 30, and 60 mg/mL): a) MIC heat map of MRSA 81 M (n=3) and b) MIC heat map of MRSA LA (n=3). Both MRSA strains were proven to have methicillin resistance via cefoxitin testing (MIC ≥8 μg/mL). All biological replicates were performed with technical duplicates. (c) Bar graph of FIC index values for analysis of the combinatory effect of the bioactive metabolites and cefoxitin. Synergy of the bioactive material was evident as the individual MICs for MRSA 81M and MRSA LA were 100 μg/mL and 60 μg/mL, respectively, and all concentrations of bioactive material (5, 30, and 60 mg/mL) elicited either an additive (FIC >0.5-1.0) or synergistic (FIC ≤0.5) effect to reduce the concentration of cefoxitin needed to elicit a growth inhibitory MIC result against the respective MRSA strains. An average of biological replicates is shown (n=3).

FIG. 2. Heat plot of the MIC percent growth inhibition of MRSA 81 M by combination testing of the β-lactam antibiotic cefoxitin (0-100 μg/mL) and bioactive metabolites (30 mg/mL) MWCO 3000 filtrate (i.e. ≤3,000 Da) and retentate (i.e. >3,000 Da) (n=3). All biological replicates were performed in technical duplicates. Only MRSA 81 M was selected for testing as it showed greater sensitivity to the bioactive material and displayed synergistic FIC values at all tested concentrations; the concentration of 30 mg/mL bioactive material was selected as it was the lowest concentration that had a FIC value well under 0.5.

FIG. 3. Heat plot of the MIC percent growth inhibition of MRSA 81 M by combination testing of the β-lactam antibiotic cefoxitin (0-100 μg/mL) and DSM13241 bioactive metabolites (30 mg/mL) MWCO 3000 filtrate (i.e. ≤3,000 Da), two washes of the retentate pellet, and retentate only (i.e. >3,000 Da) (n=3). All biological replicates were performed in technical duplicates. The 30 mg/mL bioactive material concentration was selected for fractional testing as it was the lowest tested concentration resulting in a synergistic FIC index value for an MRSA clinical strain. The two washes of the retentate that were performed also showed some residual bioactive activity.

FIG. 4. (a,b,c) Comparison of cell pellets of MRSA 81M and MRSA LA following 24 h incubation with bioactive material (30 mg/mL) at 37° C.±1° C. (cells were pelleted at 4,000 rpm centrifugation for 15 mins): (a) From left to right: untreated MRSA 81M, bioactive-treated MRSA 81M, untreated MRSA LA, and bioactive-treated MRSA LA. (b) From left to right: untreated MRSA 81 M and bioactive-treated MRSA 81 M. (c) From left to right: untreated MRSA LA and bioactive-treated MRSA LA. Interestingly, it should be noted that all cell pellets had nearly identical final CFU/mL concentrations following 24 h incubation (FIG. 5), even though untreated MRSA pellet sizes were visually much smaller and more densely compacted following centrifugation than their bioactive-treated counterparts. (d) Whisker box plot displaying the absorbance measurement at 450 nm (A₄₅₀) of extracted carotenoids from each of the two treatments described for both strains. Central black bar indicates median, and the upper and lower whisker caps show the maximum and minimum, respectively (n=3). All biological replicates were performed in technical duplicates. The golden pigment displayed by antioxidant carotenoids was significantly reduced for MRSA 81 M (U=0; p <0.05; Mann-Whitney) and MRSA LA (U=0; p <0.05; Mann-Whitney) following treatment with 30 mg/mL of DSM13241 bioactive metabolites. Remarkably, although the wildtype (i.e. untreated) MRSA LA possessed very little orange pigmentation originally, treatment with the bioactive material caused the MRSA LA to lose most of its color resulting in an almost purely white pellet (median A₄₅₀=0.0435) (U=0; p <0.05; Mann-Whitney).

FIG. 5. (a,b) Bar graph showing the average decrease of Staphylococcal survival to 1.5% v/v hydrogen peroxide for (a) MRSA 81 M (n=3) and (b) MRSA LA (n=3) before and after 1 h incubation at 37° C.±1° C. in PBS solution with and without bioactive treatment (30 mg/mL). The starting cell concentrations (T=0 h) and final surviving cell concentrations (T=1 h) for (a) MRSA 81M and (b) MRSA LA are shown. All biological replicates were performed in technical duplicates. The average CFU/mL are displayed. Error bars show standard deviation. Following incubation with 1.5% v/v hydrogen peroxide, both bioactive-treated MRSA 81 M (W=0; p <0.05; Wilcoxon signed-rank) and MRSA LA (W=0; p <0.05; Wilcoxon signed-rank) showed significant reduction in cell death at >99.99% and 99.67%, respectively. Untreated MRSA 81M (W=0; p <0.05; Wilcoxon signed-rank) also showed a minor yet statistically significant reduction in cell death (19.70%) following incubation with hydrogen peroxide; MRSA LA also showed some reduction in cell death (16.67%), however this was not deemed significant (W=5; p >0.05; Wilcoxon signed-rank).

FIG. 6. Whisker box plot depicting the percent decrease of Staphylococcal survival to 1.5% v/v hydrogen peroxide for MRSA 81 M (n=3) and MRSA LA (n=3) following 1 h incubation at 37° C.±1° C. in PBS solution with and without bioactive treatment (30 mg/mL). All biological replicates were performed in technical duplicates. Central black bar indicates median, and the upper and lower whisker caps show the maximum and minimum, respectively. Following incubation with 1.5% v/v hydrogen peroxide, both bioactive-treated MRSA 81 M (W=0; p <0.05; Wilcoxon signed-rank) and MRSA LA (W=0; p <0.05; Wilcoxon signed-rank) showed significant reduction in cell death at >99.99% and 99.67%, respectively. Untreated MRSA 81 M (W=0; p <0.05; Wilcoxon signed-rank) also showed a minor yet statistically significant reduction in cell death (19.70%) following incubation with hydrogen peroxide; MRSA LA also showed some reduction in cell death (16.67%), however this was not deemed significant (W=5; p >0.05; Wilcoxon signed-rank).

FIG. 7. Bar graph indicating the FIC Index for Staphylococcus pseudintermidius C260 22-2011 dtqa. Enterococcus faecium cell free supernatant was added at 0-120 mg/mL and the MIC for cefoxitin was measured. The data indicates a synergistic effect with cell free supernatant and cefoxitin.

FIG. 8. Heat plot of the MIC percent growth inhibition of MRSP C260 22-2011 dtqa by combination testing of the β-lactam antibiotic cefoxitin (0-250 μg/mL) and bioactive metabolites (0-120 mg/mL) (n=2). All biological replicates were performed in technical duplicates.

### SUMMARY

In accordance with an aspect, there is provided a synergistic combination comprising a quorum-sensing inhibitor and/or a postbiotic metabolite and an antibiotic.

In an aspect, the quorum-sensing inhibitor and/or postbiotic metabolite comprises a peptide, small molecule, lipid, sugar, or a combination thereof.

In an aspect, the peptide comprises or consists of one or more of the following amino acid sequences: XX[L or I]PPK, wherein X designates a hydrophobic amino acid; X₁X₂[L or I]PPK, wherein X₁ is selected from N, C, Q, M, S, and T and wherein X₂ is selected from A, I, L, and V; MALPPK; CVLPPK; HLLPLP; LKPTPEGD; YPVEPF; YPPGGP; YPPG; NQPY; LPVPK; ALPK; EVLNCLALPK; LPLP; HLLPLPL; YVPEPF; KYVPEPF; EMPFKPYPVEPF; and variants thereof altered by deletion, substitution or insertion wherein the activity of the molecules is not substantially reduced, including peptides and/or variants thereof with post-translational modifications including glycosylation.

In an aspect, the quorum-sensing inhibitor and/or postbiotic metabolite is a peptide and comprises or consists of 4, 5, 6, 7, 8, 9, 10, 11, 12, 13, 14, 15, 16, 17, 18, 19, or 20 amino acid residues, such as from 2, 3, 4, 5, 6, 7, 8, or 9 to about 3, 4, 5, 6, 7, 8, 9, or 10 amino residues.

In an aspect, the quorum-sensing inhibitor and/or postbiotic metabolite is less than about 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, or 500 Da in size, such as less than about 3000, 2000, or 1000 Da in size.

In an aspect, the quorum-sensing inhibitor and/or postbiotic metabolite is in a probiotic bacterial culture fraction, such as a supernatant.

In an aspect, the probiotic bacterial culture fraction is a cell-free spent medium (CSFM), which is optionally concentrated in liquid or dry form (e.g. by lyophilization and/or spray-drying).

In an aspect, the CSFM has a molecular weight cut-off of about 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, or 500 Da in size, such as about 3000, 2000, or 1000 Da.

In an aspect, the antibiotic is an aminoglycoside, a bacitracin, a beta-lactam antibiotic, a cephalosporin, a chloramphenicol, a glycopeptide, a macrolides, a lincosamide, a penicillin, a quinolone, a rifampin, a glycopeptide, a tetracycline, a trimethoprim, a sulfonamides, or a combination thereof.

In an aspect, the antibiotic is a β-lactam antibiotic, such as cefoxitin.

In an aspect, the quorum-sensing inhibitor and/or postbiotic metabolite is derived from the culture medium or supernatant of probiotic bacteria of the genera Aerococcus, Bacillus, Bacteroides, Bifidobacterium, Clostridium, Enterococcus, Fusobacterium, Lactobacillus, Lactococcus, Leuconostoc, Melissococcus, Micrococcus, Pediococcus, Peptostrepococcus, Propionibacterium, Staphylococcus, Streptococcus, Weissella, or combinations thereof.

In an aspect, the quorum-sensing inhibitor and/or postbiotic metabolite is derived from Lactobacillus, such as Lactobacillus acidophilus, such as Lactobacillus acidophilus (DSM13241) and/or wherein the quorum-sensing inhibitor and/or postbiotic metabolite is derived from Enterococcus, such as Enterococcus faecium.

In an aspect, the combination further comprises a probiotic.

In an aspect, the probiotic is of the genera Aerococcus, Bacillus, Bacteroides, Bifidobacterium, Clostridium, Enterococcus, Fusobacterium, Lactobacillus, Lactococcus, Leuconostoc, Melissococcus, Micrococcus, Pediococcus, Peptostrepococcus, Propionibacterium, Staphylococcus, Streptococcus, Weissella, or combinations thereof.

In an aspect, the probiotic is Lactobacillus, such as Lactobacillus acidophilus, such as Lactobacillus acidophilus (DSM13241) and/or Enterococcus, such as Enterococcus faecium.

In an aspect, the probiotic is live.

In an aspect, the probiotic is present in an amount of about 100 million to about 500 million CFU per dose, such as about 200 million CFU per dose.

In accordance with an aspect, there is provided a composition comprising the synergistic combination described herein.

In accordance with an aspect, there is provided a method for:

(a) resensitizing an antibiotic-resistant infection to an antibiotic, the method comprising administering a quorum-sensing inhibitor and/or a postbiotic metabolite to a subject afflicted with the infection;

(b) decreasing resistance of a bacterial infection to hydrogen peroxide cytotoxicity, the method comprising administering a quorum-sensing inhibitor and/or a postbiotic metabolite to a subject afflicted with the infection;

(c) treating and/or preventing diarrhea in a subject, the method comprising administering a quorum-sensing inhibitor and/or a postbiotic metabolite and an antibiotic to the subject;

(d) treating MRSA or MRSP, the method comprising administering a quorum-sensing inhibitor and/or a postbiotic metabolite and an antibiotic to a subject afflicted with MRSA or MRSP;

(e) reducing carotenoid synthesis in bacteria, the method comprising administering a quorum-sensing inhibitor and/or a postbiotic metabolite to the bacteria; and/or

(f) sensitizing bacteria to oxidant killing, the method comprising administering a quorum-sensing inhibitor and/or a postbiotic metabolite to the bacteria.

In an aspect, the antibiotic is an aminoglycoside, a bacitracin, a beta-lactam antibiotic, a cephalosporin, a chloramphenicol, a glycopeptide, a macrolides, a lincosamide, a penicillin, a quinolone, a rifampin, a glycopeptide, a tetracycline, a trimethoprim, a sulfonamides, or a combination thereof.

In an aspect, the antibiotic is a β-lactam antibiotic, such as cefoxitin.

In an aspect, the quorum-sensing inhibitor and/or postbiotic metabolite comprises a peptide, small molecule, lipid, sugar, or a combination thereof.

In an aspect, the peptide comprises or consists of one or more of the following amino acid sequences: XX[L or I]PPK, wherein X designates a hydrophobic amino acid; X₁X₂[L or I]PPK, wherein X₁ is selected from N, C, Q, M, S, and T and wherein X₂ is selected from A, I, L, and V; MALPPK; CVLPPK; HLLPLP; LKPTPEGD; YPVEPF; YPPGGP; YPPG; NQPY; LPVPK; ALPK; EVLNCLALPK; LPLP; HLLPLPL; YVPEPF; KYVPEPF; EMPFKPYPVEPF; and variants thereof altered by deletion, substitution or insertion wherein the activity of the molecules is not substantially reduced, including peptides and/or variants thereof with post-translational modifications including glycosylation.

In an aspect, the quorum-sensing inhibitor and/or postbiotic metabolite is a peptide and comprises or consists of 4, 5, 6, 7, 8, 9, 10, 11, 12, 13, 14, 15, 16, 17, 18, 19, or 20 amino acid residues, such as from 2, 3, 4, 5, 6, 7, 8, or 9 to about 3, 4, 5, 6, 7, 8, 9, or 10 amino residues.

In an aspect, the quorum-sensing inhibitor and/or postbiotic metabolite is less than about 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, or 500 Da in size, such as less than about 3000, 2000, or 1000 Da in size.

In an aspect, the quorum-sensing inhibitor and/or postbiotic metabolite is in a probiotic bacterial culture fraction, such as a supernatant.

In an aspect, the probiotic bacterial culture fraction is a cell-free spent medium (CSFM), which is optionally concentrated in liquid or dry form (e.g. by lyophilization and/or spray-drying).

In an aspect, the CSFM has a molecular weight cut-off of about 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, or 500 Da in size, such as about 3000, 2000, or 1000 Da.

In an aspect, the quorum-sensing inhibitor and/or postbiotic metabolite is derived from the culture medium or supernatant of probiotic bacteria of the genera Aerococcus, Bacillus, Bacteroides, Bifidobacterium, Clostridium, Enterococcus, Fusobacterium, Lactobacillus, Lactococcus, Leuconostoc, Melissococcus, Micrococcus, Pediococcus, Peptostrepococcus, Propionibacterium, Staphylococcus, Streptococcus, Weissella, or combinations thereof.

In an aspect, the quorum-sensing inhibitor and/or postbiotic metabolite is derived from Lactobacillus, such as Lactobacillus acidophilus, such as Lactobacillus acidophilus (DSM13241) and/or wherein the quorum-sensing inhibitor and/or postbiotic metabolite is derived from Enterococcus, such as Enterococcus faecium.

In an aspect, the method further comprises administering a probiotic to the subject.

In an aspect, the probiotic is of the genera Aerococcus, Bacillus, Bacteroides, Bifidobacterium, Clostridium, Enterococcus, Fusobacterium, Lactobacillus, Lactococcus, Leuconostoc, Melissococcus, Micrococcus, Pediococcus, Peptostrepococcus, Propionibacterium, Staphylococcus, Streptococcus, Weissella, or combinations thereof.

In an aspect, the probiotic is Lactobacillus, such as Lactobacillus acidophilus, such as Lactobacillus acidophilus (DSM13241) and/or wherein the quorum-sensing inhibitor and/or postbiotic metabolite is derived from Enterococcus, such as Enterococcus faecium.

In an aspect, the probiotic is live.

In an aspect, the probiotic is present in an amount of about 100 million to about 500 million CFU per dose, such as about 200 million CFU per dose.

In an aspect, the subject is a pet, such as a dog.

In an aspect, the subject is a farm animal, such as swine or poultry.

In an aspect, the subject is a human.

In an aspect, the quorum-sensing inhibitor and/or postbiotic metabolite and the antibiotic act synergistically.

In accordance with an aspect, there is provided a tablet or capsule comprising the synergistic combination or the composition described herein.

In accordance with an aspect, there is provided a use of the synergistic combination or the composition described herein in the method of described herein.

In an aspect, the synergistic combination or the composition described herein is for use in the method described herein.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from the detailed description.

### DETAILED DESCRIPTION

Postbiotic metabolites have been described for use in treating various types of infections in International Patent Application Publication Nos. WO 2009/155711, WO 2015/021530, 2018/165764, and WO 2018/165765. Each of these references is incorporated herein by reference in its entirety at least with respect to the teachings of specific postbiotic metabolites, their methods of identification, their bacterial sources, and infections in which they find use in treating.

It has now been found that quorum-sensing inhibitors and/or postbiotic metabolites synergistically treat infections when combined with antibiotics and/or are capable of affecting antibiotic resistance. Further, probiotic cell-free spent medium (CSFM), including MWCO 3000-filtered CSFM, containing quorum-sensing inhibitors, including postbiotic metabolites as well as the quorum-sensing inhibitors and postbiotic metabolites themselves synergistically treat infections when combined with antibiotics. Moreover, the CSFM, quorum-sensing inhibitors, and/or postbiotic metabolites in aspects are capable of overcoming antibiotic resistance and can resensitize antibiotic-resistant bacteria to conventional antibiotics. In other aspects, the CSFM, quorum-sensing inhibition molecules, and/or postbiotic metabolites described herein are capable of decreasing resistance of a bacterial infection to hydrogen peroxide cytotoxicity.

Furthermore, a number of compositions have been tested in specific populations and effective doses and combinations have been determined. In particular, veterinary dosing and scheduling is described herein.

### Definitions

Unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N.Y. 1994); Sambrook et al., Molecular Cloning. A Laboratory Manual, Cold Springs Harbor Press (Cold Springs Harbor, N Y 1989), each of which are incorporated herein by reference. For the purposes of the present invention, the following terms are defined below.

By “derived,” it is meant that the molecules are either directly or indirectly produced by the probiotic bacteria. For example, the probiotic bacteria may secrete the molecules directly into the culture medium. In other aspects, the molecules can be formed indirectly within the culture medium, for example, by being cleaved from longer peptides, or may be small molecules, sugars, lipids, etc. either secreted into or found/produced in the culture medium of probiotic bacteria.

“Isolated” refers to a molecule that has been purified from its source or has been prepared by recombinant or synthetic methods and purified. Purified proteins are substantially free of other amino acids. “Substantially free” herein means less than about 5%, typically less than about 2%, more typically less than about 1%, even more typically less than about 0.5%, most typically less than about 0.1% contamination with other source amino acids.

An “essentially pure” composition means a composition comprising at least about 90% by weight of the molecule in question, based on total weight of the composition, typically at least about 95% by weight, more typically at least about 90% by weight, even more typically at least about 95% by weight, and even more typically at least about 99% by weight, based on the total weight of the composition.

As used herein, “treatment” or “therapy” is an approach for obtaining beneficial or desired clinical results. For the purposes described herein, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” and “therapy” can also mean prolonging survival as compared to expected survival if not receiving treatment or therapy. Thus, “treatment” or “therapy” is an intervention performed with the intention of altering the pathology of a disorder. Specifically, the treatment or therapy may directly prevent, slow down or otherwise decrease the pathology of a disease or disorder such as an infection, or may render the infection more susceptible to treatment or therapy by other therapeutic agents.

The terms “therapeutically effective amount”, “effective amount” or “sufficient amount” mean a quantity sufficient, when administered to a subject, including a mammal, for example a human, to achieve a desired result, for example an amount effective to treat an infection. Effective amounts of the molecules described herein may vary according to factors such as the disease state, age, sex, and weight of the subject. Dosage or treatment regimes may be adjusted to provide the optimum therapeutic response, as is understood by a skilled person.

Moreover, a treatment regime of a subject with a therapeutically effective amount may consist of a single administration, or alternatively comprise a series of applications. The length of the treatment period depends on a variety of factors, such as the severity and/or site of the disease, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The molecules described herein may, in aspects, be administered before, during or after treatment with conventional therapies for the disease or disorder in question, such as an infection.

The term “subject” as used herein refers to any member of the animal kingdom, including birds, fish, invertebrates, amphibians, mammals, and reptiles. Typically, the subject is a human or non-human vertebrate. Non-human vertebrates include livestock animals, companion animals, and laboratory animals. Non-human subjects also specifically include non-human primates as well as rodents. Non-human subjects also specifically include, without limitation, poultry, chickens, horses, cows, pigs, goats, dogs, cats, guinea pigs, hamsters, mink, rabbits, crustaceans, and molluscs. Typically the subject is poultry or a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known pharmaceutically acceptable additive, excipient, diluent, carrier, and the like. Typically, a composition consisting essentially of a set of components will comprise less than 5% by weight, typically less than 3% by weight, more typically less than 1% by weight of non-specified components.

It will be understood that any component defined herein as being included may be explicitly excluded from the claimed invention by way of proviso or negative limitation. For example, in aspects, the molecules described herein are not bacteriocins.

In addition, all ranges given herein include the end of the ranges and also any intermediate range points, whether explicitly stated or not.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### Quorum-Sensing Inhibitors and Compositions Comprising Quorum-Sensing Inhibitors

The present invention provides quorum-sensing inhibitors and postbiotic metabolites as well as culture fractions, such as cell-free spent medium (CSFM), derived from probiotic bacteria. The molecules described herein in aspects may minimize, inhibit, treat, and/or prevent infection in a subject, may resensitize an antibiotic-resistant bacteria to an antibiotic to which it was previously resistant, or may act synergistically with an antibiotic to minimize, inhibit, treat, and/or prevent an infection. The molecules in alternate or additional aspects may act to decrease resistance of a bacterial infection to hydrogen peroxide cytotoxicity. Combinations of these molecules are also considered, for example wherein one such molecule may be a peptide and one may be another type of molecule, such as a lipid, sugar, small molecule, etc. Combinations of peptide molecules are considered as well in the compositions described herein.

In aspects, the molecules are small molecules, typically proteinaceous, that are temperature resistant (can be heated, frozen and thawed and still exhibit activity), are stable for long periods of time frozen (over two years), can be produced readily in large volumes (for example about 2 mg/L), can be concentrated, and/or can be dried by methods such as lyophilisation and/or spray-drying. The molecules may be, in aspects, proteins, small peptides, small molecules, lipids, sugars, and so on. There may be combinations of such molecules that work in concert to achieve the effects described herein. The molecules are typically found in the supernatant of probiotic bacteria cultures as described herein and the supernatant can be provided as a CSFM or as a CSFM fraction with a specific molecular weight cut off about 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, or 500 Da, such as about 3000, 2000, or 1000 Da, meaning that the molecules are less than about 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, or 500 Da in size, such as less than about 3000, 2000, or 1000 Da in size.

Typically, the molecules are peptides and the peptides are typically from about 2 to about 10 amino acid residues in length. The molecules described herein are often administered as a concentrated CFSM from a probiotic bacterial culture. It will be understood that any of the specific molecules described in International Patent Application Publication Nos. WO 2009/155711, WO 2015/021530, 2018/165764, and WO 2018/165765 are incorporated herein by reference.

Briefly, the molecules include, for example, peptides comprising or consisting of one or more of the following amino acid sequences: MALPPK, CVLPPK, HLLPLP, and LKPTPEGD. It is understood by one of skill in the art that these sequences can be altered by deletion, substitution or insertion so long as the activity of the molecules is not substantially reduced. For example, the sequence may comprise or consist of XX[L or I]PPK, wherein X designates a hydrophobic amino acid. Alternatively, the sequence may comprise or consist of X₁X₂[L or I]PPK, wherein X₁ is selected from N, C, Q, M, S, and T and wherein X₂ is selected from A, I, L, and V. Further, the molecules include, for example, peptides comprising or consisting of one or more of the following amino acid sequences: YPVEPF, YPPGGP, YPPG, NQPY, LPVPK, ALPK, EVLNCLALPK, LPLP, HLLPLPL, YVPEPF, KYVPEPF, and EMPFKPYPVEPF. Typically, the peptide comprises or consists of 4, 5, 6, 7, 8, 9, 10, 11, 12, 13, 14, 15, 16, 17, 18, 19, or 20 amino acid residues, typically from 2, 3, 4, 5, 6, 7, 8, or 9 to about 3, 4, 5, 6, 7, 8, 9, or 10 amino residues. It will be understood that the molecules may be used in any combination, isolated individually or collectively, or as produced by probiotic bacteria in the culture medium. The culture medium, for example, CFSM, may be provided in the form of a concentrated liquid or powder, for example.

The molecules can further have insertions, substitutions, or deletions of one or more of the amino acid residues. Furthermore, the molecules described herein may further be altered with glycosylation, unglycosylation, organic and inorganic salts and covalently modified. Also encompassed are molecules modified to increase in vivo half-life, e.g., PEGylated. Possible but non-limiting modifications to the molecules described herein include modifications comprising combinations of amino acid substitutions together with a deletion of one or more amino acids or the addition of one or more amino acids.

In particular, any probiotic bacterial species may serve as the source of the molecules described herein, including for example, the genera Aerococcus, Bacillus, Bacteroides, Bifidobacterium, Clostridium, Enterococcus, Fusobacterium, Lactobacillus, Lactococcus, Leuconostoc, Melissococcus, Micrococcus, Pediococcus, Peptostrepococcus, Propionibacterium, Staphylococcus, Streptococcus, Weissella, or combinations thereof.

Specific probiotically active lactic acid bacterial species include, for example, Enterococcus faecalis, Enterococcus faecium, Lactobacillus acidophilus, Lactobacillus alimentarius, Lactobacillus casei Shirota, Lactobacillus casei subsp. paracasei, Lactobacillus casei subsp. casei, Lactobacillus casei, Lactobacillus crispatus, Lactobacillus curvatus, Lactobacillus delbruckii subsp. lactis, Lactobacillus delbrueckii subsp. bulgaricus, Lactobacillus farciminus, Lactobacillus fermentum, Lactobacillus gasseri, Lactobacillus helveticus, Lactobacillus johnsonii, Lactobacillus paracasei subsp. paracasei, Lactobacillus rhamnosus, Lactobacillus plantarum, Lactobacillus reuteri, Lactobacillus rhamnosus, Lactobacillus sake, Lactococcus lactis, Lactocoocus lactis subsp. cremoris, Streptococcus faecalis, Streptococcus faecium, Streptococcus salivarius and Streptococcus thermophilus. Further examples comprise probiotically active Bifidobacterium species including Bifidobacterium infantis, Bifidobacterium adolescentis, Bifidobacterium bifidum, Bifidobacterium longum, Bifidobacterium lactis, Bifidobacterium animalis, Bifidobacterium breve, and combinations thereof

Further probiotic bacterial species include, for example, probiotically active Paenibacillus lautus, Bacillus coagulans, Bacillus licheniformis, Bacillus subtilis, Micrococcus varians, Pediococcus acidilactici, Pediococcus pentosaceus, Pediococcus acidi-lactici, Pediococcus halophilus, Staphylococcus carnosus and Staphylococcus xylosus, as well as the microorganism Lactobacillus casei ssp. rhamnosus strain LC-705, DSM 7061 described in EP publication No. 0576780, and described as Lactobacillus rhamnosus LC-705, DSM 7061 in U.S. Pat. No. 5,908,646, alone or in combination with a bacterium of the genus Propionibacterium or another strain of Lactobacillus casei.

Specific probiotic bacterial strains that may produce the molecules described herein include, for example, Bifidobacterium animalis strain DSM15954, Bifidobacterium longum subsp. infantis strain DSM15953, Bifidobacterium longum subsp. longum strain DSM15955, Enterococcus faecium strain DSM15958, Lactobacillus acidophilus strain DSM13241 (also referred to as La-5 or La-21), Lactobacillus delbrueckii subsp. bulgaricus strain DSM15956, Lactobacillus helveticus strain DSM14998, Lactobacillus helveticus strain DSM14997, Lactococcus lactis strain DSM14797, Streptococcus thermophilus strain DSM15957, Lactobacillus fermentum strain ATCC55845, Lactobacillus rhamnosus strain ATCC55826, and combinations thereof.

In typical aspects, the molecules are derived from, for example, Lactobacillus acidophilus, including the strain DSM13241, strains of Pediococcus, strains of Bifidobacterium such as but not limited to Bifidobacterium longum, Bifidobacterium bifidum, Bifidobacterium infantis, and Bifidobacterium crudilactis, Lactobacillus fermentum, Lactobacillus rhamnosus, Lactobacillus helveticus, Lactobacillus plantarum, Lactococcus lactis, Streptococcus thermophiles, and combinations thereof.

Likewise, any known antibiotic may be used in combination with the molecules described herein, before, after, and/or during administration of the molecules. General classes of antibiotics include, for example, aminoglycosides, bacitracin, beta-lactam antibiotics, cephalosporins, chloramphenicol, glycopeptides, macrolides, lincosamides, penicillins, quinolones, rifampin, glycopeptide, tetracyclines, trimethoprim and sulfonamides. In some aspects, the administrations of a combination of molecules and antibiotics are spaced sufficiently close together such that a synergistic effect is achieved.

Exemplary antibiotics within the classes recited above are provided as follows. Any of these may be used individually or in various combinations. Exemplary aminoglycosides include Streptomycin, Neomycin, Framycetin, Parpmycin, Ribostamycin, Kanamycin, Amikacin, Dibekacin, Tobramycin, Hygromycin B, Spectinomycin, Gentamicin, Netilmicin, Sisomicin, Isepamicin, Verdamicin, Amikin, Garamycin, Kantrex, Netromycin, Nebcin, and Humatin. Exemplary carbacephems include Loracarbef (Lorabid). Exemplary carbapenems include Ertapenem, lnvanz, Doripenem, Finibax, Imipenem/Cilastatin, Primaxin, Meropenem, and Merrem. Exemplary cephalosporins include Cefadroxil, Durisef, Cefazolin, Ancef, Cefalotin, Cefalothin, Keflin, Cefalexin, Keflex, Cefaclor, Ceclor, Cefamandole, Mandole, Cefoxitin, Mefoxin, Cefprozill, Cefzil, Cefuroxime, Ceftin, Zinnat, Cefixime, Suprax, Cefdinir, Omnicef, Cefditoren, Spectracef, Cefoperazone, Cefobid, Cefotaxime, Claforan, Cefpodoxime, Fortaz, Ceftibuten, Cedax, Ceftizoxime, Ceftriaxone, Rocephin, Cefepime, Maxipime, and Ceftrobriprole. Exemplary glycopeptides include Dalbavancin, Oritavancin, Teicoplanin, Vancomycin, and Vancocin. Exemplary macrolides include Azithromycin, Sithromax, Sumamed, Zitrocin, Clarithromycin, Biaxin, Dirithromycin, Erythromycin, Erythocin, Erythroped, Roxithromycin, Troleandomycin, Telithromycin, Ketek, and Spectinomycin. Exemplary monobactams include Aztreonam. Exemplary penicillins include Amoxicillin, Novamox, Aoxil, Ampicillin, Azlocillin, Carbenicillin, Coxacillin, Diloxacillin, Flucloxacillin Floxapen, Mezlocillin, Methicillin, Nafcillin, Oxacillin, Penicillin, and Ticarcillin. Exemplary polypeptides include Bacitracin, Colistin, and Polymyxin B. Exemplary quinolones include Ciprofloxacin, Cipro, Ciproxin, Ciprobay, Enoxacin, Gatifloxacin, Tequin, Levofloxacin, Levaquin, Lomefloxacin, Moxifloxacin, Avelox, Norfloxacin, Noroxin, Ofloxacin, Ocuflox, Trovafloxacin, and Trovan. Exemplary sulfonamides include Mefenide, Prontosil, Sulfacetamide, Sulfamethizole, Sulfanilamide, Sulfasalazine, Sulfisoxazole, Trimethoprim, Trimethoprim-Sulfamethoxazole (co-trimoxazole), and Bactrim. Exemplary tetracyclines include Demeclocyline, Doxycycline, Vibramycin, Minocycline, Minocin, Oxytetracycline, Terracin, Tetracycline, and Sumycin. Other exemplary antibiotics include Salvarsan, Chloamphenicol, Chloromycetin, Clindamycin, Cleocin, Linomycin, Ethambutol, Fosfomycin, Fusidic Acid, Fucidin, Furazolidone, Isoniazid, Linezolid, Zyvox, Metronidazole, Flagyl, Mupirocin, Bactroban, Nitrofurantion, Macrodantin, Macrobid, Platensimycin, Pyrazinamide, Quinupristin/Dalfopristin (Syncerid), Rifampin (rifampicin), and Tinidazole. In some aspects, the exemplary antibiotics include xylitol, hydrogen peroxide (whether derived from immune cells or other sources), chlorhexidine, delmopinol, decapinol, hopchlorite, chlorine dioxide and cetylpyridinium chloride.

The molecules can be incorporated into a variety of substances for administration to a subject such as any type of animal and humans. For example, the molecules can be incorporated into any type of food product, nutritional supplement or beverage for animal or human consumption, including animal feed or drink.

As a therapeutic, the molecules described herein can be administered in a manner to an animal or human for the effective treatment of infection, including resensitizing the infection for treatment by a conventional antibiotic and/or decreasing resistance of a bacterial infection to hydrogen peroxide cytotoxicity. As a therapeutic or prophylactic, the treatment can be in conjunction with other therapies as is desired. In another embodiment, the molecules described herein can be used in compositions and in methods in addition to use of whole probiotic bacteria. Alternatively, whole probiotic bacteria can be used alone, provided the bacteria are cultured and/or used such that the molecules are produced in the culture medium in a therapeutically effective amount.

In a generalized aspect, the molecules described herein can be provided in a therapeutically effective amount alone or within a composition and in amounts that may vary according to factors such as the infection state/health, age, sex, and weight of the recipient. Dosage regimes may be adjusted to provide the optimum therapeutic response and may be at the discretion of the attending physician or veterinarian. For example, several divided doses may be administered daily or on at periodic intervals, and/or the dose may be proportionally reduced as indicated by the exigencies of the therapeutic situation. The amount of the molecule for administration will depend on the route of administration, time of administration and may be varied in accordance with individual subject responses. Suitable administration routes are, for example, via the topical, oral, rectal or parenteral (e.g., intravenous, subcutaneous or intramuscular) route. In addition, the molecules can be incorporated into polymers allowing for sustained release, the polymers being implanted in the vicinity of where delivery is desired, for example, at the site of an infection, or the polymers can be implanted, for example, subcutaneously or intramuscularly or delivered intravenously or intraperitoneally to result in systemic delivery of the molecules described herein.

The molecules described herein can be administered in the form of, for example, a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch. The molecules may be administered as a cell-free supernatant, which, in aspects is a cell-free supernatant concentrate. The concentrate may be in liquid or powder form.

The formulations include those suitable for oral, rectal, nasal, inhalation, topical (including dermal, transdermal, buccal and sublingual), vaginal, parenteral (including subcutaneous, intramuscular, intravenous, intradermal, intraocular, intratracheal, and epidural), intramammary, or inhalation administration. The formulations can conveniently be presented in unit dosage form and can be prepared by conventional pharmaceutical techniques. Such techniques include the step of bringing into association the active ingredient and a pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion, etc.

A tablet may be made by compression or molding, optionally with one or more accessory ingredients. Compressed tablets may be prepared by compressing, in a suitable machine, the molecules described herein in a free-flowing form such as a powder or granules, optionally mixed with a binder, lubricant, inert diluent, preservative, surface-active and/or dispersing agent. Molded tablets may be made by molding, in a suitable machine, a mixture of the powdered compound moistened with an inert liquid diluent. The tablets may optionally be coated or scored and may be formulated so as to provide a slow or controlled release of the active ingredient therein.

Formulations suitable for topical administration in the mouth include lozenges comprising the ingredients in a flavored base, typically sucrose and acacia or tragacanth; pastilles comprising the active ingredient in an inert base such as gelatin and glycerin, or sucrose and acacia; and mouthwashes comprising the ingredient to be administered in a suitable liquid carrier.

Formulations suitable for topical administration to the skin may be presented as ointments, creams, gels, or pastes comprising the ingredient to be administered in a pharmaceutical acceptable carrier. In one embodiment the topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing, in addition to the active ingredient, ingredients such as carriers as are known in the art to be appropriate.

Formulations suitable for inhalation may be presented as mists, dusts, powders or spray formulations containing, in addition to the active ingredient, ingredients such as carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Formulations suitable for parenteral administration include particulate preparations of the anti-angiogenic agents, including, but not limited to, low-micron, or nanometer (e.g. less than 2000 nanometers, typically less than 1000 nanometers, most typically less than 500 nanometers in average cross section) sized particles, which particles are comprised of the molecules described herein alone or in combination with accessory ingredients or in a polymer for sustained release. The formulations may be presented in unit-dose or multi-dose containers, for example, sealed ampules and vials, and may be stored in freeze-dried (lyophilized) conditions requiring only the addition of a sterile liquid carrier, for example, water for injections, immediately prior to use. Extemporaneous injection solutions and suspensions may be prepared from sterile powders, granules and tablets of the kinds previously described.

Compositions comprising the molecules described herein may comprise about 0.00001% to about 99% by weight of the active and any range there-in-between. For example, typical doses may comprise from about 0.1 μg to about 100 μg of the molecules described herein per 300 mg dose, such as about 0.5 μg, about 1 μg, about 2 μg, about 3 μg, about 4 μg, about 5 μg, about 6 μg, about 7 μg, about 8 μg, about 9 μg, about 10 μg, about 25 μg, about 50 μg, or about 75 μg per 300 mg dose, such as from about 0.1 μg to about 10 μg, or from about 1 μg to about 5 μg, or from about 1 μg to about 2 μg per 300 mg dose (and all related increments and percentages by weight).

The molecules may be administered over a period of hours, days, weeks, or months, depending on several factors, including the severity of the infection being treated, whether a recurrence of the infection is considered likely, or to prevent infection, etc. The administration may be constant, e.g., constant infusion over a period of hours, days, weeks, months, etc. Alternatively, the administration may be intermittent, e.g., the molecules may be administered once a day over a period of days, once an hour over a period of hours, or any other such schedule as deemed suitable.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in “Handbook of Pharmaceutical Additives” (compiled by Michael and Irene Ash, Gower Publishing Limited, Aldershot, England (1995)). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456 (the entirety of which is incorporated herein by reference).

Pharmaceutically acceptable carriers are well known to those skilled in the art and include, for example, sterile saline, lactose, sucrose, calcium phosphate, gelatin, dextrin, agar, pectin, peanut oil, olive oil, sesame oil and water. Furthermore the pharmaceutical composition may comprise one or more stabilizers such as, for example, carbohydrates including sorbitol, mannitol, starch, sucrose, dextrin and glucose, proteins such as albumin or casein, and buffers like alkaline phosphates.

In another non-limiting aspect, administration of the molecules can be accomplished by any method likely to introduce the molecules into the digestive tract, such as orally or rectally, after which the molecules enter the bloodstream and/or act directly on gut microbes. The bacteria producing the molecules and/or the isolated molecules can be mixed with a carrier and applied to liquid or solid feed or to drinking water. The carrier material should be non-toxic to the animal. The bacteria producing the molecules and/or the isolated molecules can also be formulated into a composition provided as an inoculant paste to be directly injected into an animal's mouth. The formulation can include added ingredients to improve palatability, improve shelf-life, impart nutritional benefits, and the like. If a reproducible and measured dose is desired, the molecules can be administered by a rumen cannula, as described herein. The amount of the molecules to be administered is governed by factors affecting efficacy. By monitoring the infection before, during and after administration of the molecules, those skilled in the art can readily ascertain the dosage level needed to reduce the amount of infection carried by the animals and/or to resensitize an antibiotic-resistant bacterial infection to an antibiotic and/or to decrease resistance of a bacterial infection to hydrogen peroxide cytotoxicity, for example. The molecules from one or more strains of probiotic bacteria can be administered together. A combination of strains can be advantageous because individual animals may differ as to the strain which is most effective.

The methods for administering the molecules are essentially the same, whether for prevention or treatment. Therefore, the need to first determine whether a pathogenic infection is being carried by the animals is removed. By routinely administering an effective dose to all the animals of a herd, the risk of contamination by a pathogenic infection can be substantially reduced or eliminated by a combination of prevention and treatment.

It is understood by one of skill in the art that the molecules and culture fractions containing such, can be used in conjunction with known therapies for prevention and/or treatment of infections in a subject. It is also understood that compositions of the molecules described herein, whether isolated or in a culture fraction or in conjunction with probiotic bacteria and/or an antibiotic, can also be used in conjunction (formulated with) with a sugar source such as for example glucose in amounts of up to about 0.01% to about 0.1% or more by weight of the composition.

It is also understood that although the compositions described herein may be directly ingested or used as an additive in conjunction with foods, it will be appreciated that they may be incorporated into a variety of foods and beverages including but not limited to yoghurts, ice creams, cheeses, baked products such as bread, biscuits and cakes, dairy and dairy substitute foods, confectionery products, edible oil compositions, spreads, breakfast cereals, juices, meats, produce, and the like. Within the scope of the term “foods” are to be included in particular food likely to be classified as functional foods, i.e. “foods that are similar in appearance to conventional foods and are intended to be consumed as part of a normal diet, but have been modified to physiological roles beyond the provision of simple nutrient. Similarly, the compositions described herein may be presented in dosage forms such as in a capsule or a dried and compressed tablet or rectal or vaginal suppository, or as an aerosol or inhaler. Again, amounts of the active molecules will vary depending on the particular food or beverage and may contain any amount up to about 100% of the product, especially when formulated as an ingestible capsule/tablet.

It is also understood by one of skill in the art that the molecules described herein, whether isolated or provided as within a culture fraction, can be combined with the use of probiotic bacteria in methods of treatment or for nutritional supplementation. In particular aspects, the molecules described herein may be combined with live probiotic bacteria of the species from which the molecules are derived. In other aspects, these bacterial species may be excluded from the compositions. In other aspects, the molecules described herein may be combined with live probiotic bacteria of a species that does not produce the molecules.

### Methods of Use

Unexpectedly, it has been found that the molecules described herein, whether administrated in isolated form or in the form of bacteria from which the molecules may be derived, find use in treating infections, in aspects enteric or non-enteric infections, a number of which are specifically described below.

In particular aspects, the molecules described herein interact synergistically with one another and/or with antibiotics or other anti-infective agents to treat and/or prevent an enteric or non-enteric infection and/or to reduce the virulence of an enteric or non-enteric infection, including reducing antibiotic resistance and/or increasing the sensitivity of a particular pathogenic microorganism to a conventional treatment such as an antibiotic.

From the above, it is evident that the molecules described herein can find use in the treatment of a wide variety of pathogens, including bacteria, viruses, yeast, fungus, and parasites. In aspects, the pathogen is enteric or non-enteric and/or the infection is at an enteric or non-enteric site.

For example, the molecules described herein may be useful in treating a bacterial infection from a genus selected from the group consisting of Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anaerorhabdus, “Anguillina”, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delfiia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filif actor, Flavimonas, Flavobacterium, Flexispira, Francisella, Fusobacterium, Gardnerella, Gemella Globicatella, Gordona, Haemophilus, Hafnia, Helicobacter, Helococcus, Holdemania, lgnavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Plesiomonas Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Selenomonas, Serpulina, Serratia, Shewenella, Shigella, Simkania, Slackia, Sphingobacterium, Sphingomonas, Spirillum, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsákamurella, Turicella, Ureaplasma, Vagococcus, Veillonella, Vibrio, Weeksella, Wolinella, Xanthomonas, Xenorhabdus, Yersinia and Yokenella.

For example, the bacterial infection may be caused by a bacterium selected from the group consisting of Actimomyces europeus, Actimomyces georgiae, Actimomyces gerencseriae, Actimomyces graevenitzii, Actimomyces israelii, Actimomyces meyeri, Actimomyces naeslundii, Actimomyces neuii Actimomyces neuii anitratus, Actimomyces odontolyticus, Actimomyces radingae, Actimomyces turicensis, Actimomyces viscosus, Arthrobacter creatinolyticus, Arthrobacter cumminsii, Arthrobacter woluwensis, Bacillus anthracis, Bacillus cereus, Bacillus circulans, Bacillus coagulans, Bacillus licheniformis, Bacillus megaterium, Bacillus myroides, Bacillus pumilus, Bacillus sphaericus, Bacillus subtilis, Bacillus thuringiensis, Borrelia afzelii, Borrelia andersonii, Borrelia bissettii, Borrelia burgdorferi, Borrelia garinii, Borrelia japonica, Borrelia lusitaniae, Borrelia tanukii, Borrelia turdi, Borrelia valaisiana Borrelia caucasica, Borrelia crocidurae, Borrelia recurrentis, Borrelia duttoni, Borrelia graingeri, Borrelia hermsii, Borrelia hispanica, Borrelia latyschewii, Borrelia mazzottii, Borrelia parked Borrelia persica, Borrelia recurrentis, Borrelia turicatae, Borrelia venezuelensi, Bordetella bronchiseptica, Bordetella Bordetella holmseii, Bordetella parapertussis, Bordetella pertussis, Bordetella trematum, Clostridium absonum, Clostridium argentinense, Clostridium baratii, Clostridium bifermentans, Clostridium beijerinckii, Clostridium butyricum, Clostridium cadaveris, Clostridium camis, Clostridium celatum, Clostridium clostridioforme, Clostridium cochlearium, Clostridium cocleatum, Clostridium difficile, Clostridium fallax, Clostridium ghonii, Clostridium glycolicum, Clostridium haemolyticum, Clostridium hastiforme, Clostridium histolyticum, Clostridium indolis, Clostridium innocuum, Clostridium irregulare, Clostridium leptum, Clostridium limosum, Clostridium malenominatum, Clostridium novyi, Clostridium oroticum, Clostridium paraputriβcum, Clostridium perfringens, Clostridium piliforme, Clostridium putrefasciens, Clostridium ramosum, Clostridium septicum, Clostridium sordelii, Clostridium sphenoides, Clostridium sporogenes, Clostridium subterminale, Clostridium symbiosum, Clostridium tertium, Escherichia coli, Escherichia fergusonii, Escherichia hermanii, Escherichia vulneris, Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus, Enterococcus solitarius, Haemophilus aegyptius, Haemophilus aphrophilus, Haemophilus par aphrophilus, Haemophilus parainfluenzae, Haemophilus segnis, Haemophilus ducreyi, Haemophilus influenzae, Klebsiella omitholytica, Klebsiella oxytoca, Klebsiella planticola, Klebsiella pneumoniae, Klebsiella ozaenae, Klebsiella terrigena, Lysteria ivanovii, Lysteria monocytogenes, Mycobacterium abscessus, Mycobacterium africanurn, Mycobacterium alvei, Mycobacterium asiaticum, Mycobacterium aurum, Mycobacterium avium, Mycobacterium bohemicum, Mycobacterium bovis, Mycobacterium branded, Mycobacterium brumae, Mycobacterium celatum, Mycobacterium chelonae, Mycobacterium chubense, Mycobacterium confluentis, Mycobacterium conspicuum, Mycobacterium cookii, Mycobacterium flavescens, Mycobacterium fortuitum, Mycobacterium gadium, Mycobacterium gastri, Mycobacterium genavense, Mycobacterium gordonae, Mycobacterium goodii, Mycobacterium haemophilum, Mycobacterium hassicum, Mycobacterium intracellulare, Mycobacterium interjectum, Mycobacterium heidelberense, Mycobacterium kansasii, Mycobacterium lentiflavum, Mycobacterium leprae, Mycobacterium malmoense, Mycobacterium marinurn, Mycobacterium microgenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium smegmatis, Mycobacterium szulgai, Mycobacterium terrae, Mycobacterium thermoresistabile, Mycobacterium triplex, Mycobacterium triviale, Mycobacterium tuberculosis, Mycobacterium tusciae, Mycobacterium ulcerans, Mycobacterium vaccae, Mycobacterium wolinskyi, Mycobacterium xenopi, Mycoplasma buccale, Mycoplasma faucium, Mycoplasma fermentans, Mycoplasma genitalium, Mycoplasma hominis, Mycoplasma lipophilum, Mycoplasma orale, Mycoplasma penetrans, Mycoplasma pirum, Mycoplasma pneumoniae, Mycoplasma primatum, Mycoplasma salivarium, Mycoplasma spermatophilum, Pseudomonas aeruginosa, Pseudomonas alcaligenes, Pseudomonas chlororaphis, Pseudomonas fluorescens, Pseudomonas luteola. Pseudomonas mendocina, Pseudomonas monteilii, Pseudomonas oryzihabitans, Pseudomonas pertocinogena, Pseudomonas pseudalcaligenes, Pseudomonas putida, Pseudomonas stutzeri, Rickettsia africae, Rickettsia akari, Rickettsia australis, Rickettsia conorii, Rickettsia felis, Rickettsia honei, Rickettsia japonica, Rickettsia mongolotimonae, Rickettsia prowazeldi, Rickettsia rickettsiae, Rickettsia sibirica, Rickettsia slovaca, Rickettsia typhi, Salmonella choleraesuis choleraesuis, Salmonella choleraesuis arizonae, Salmonella choleraesuis bongori, Salmonella choleraesuis diarizonae, Salmonella choleraesuis houtenae, Salmonella choleraesuis indica, Salmonella choleraesuis salamae, Salmonella enteritidis, Salmonella typhi, Salmonella typhimurium, Shigella boydii, Shigella dysentaeriae, Shigella flexneri, Shigella sonnet Staphylococcus aureus, Staphylococcus auricularis, Staphylococcus capitis capitis, Staphylococcus c. ureolyticus, Staphylococcus caprae, Staphylococcus aureus, Staphylococcus cohnii cohnii, Staphylococcus c. ureolyticus, Staphylococcus epidermidis, Staphylococcus pseudintermedius, Staphylococcus equorum, Staphylococcus gallinarum, Staphylococcus haemolyticus, Staphylococcus hominis hominis, Staphylococcus h. novobiosepticius, Staphylococcus hyicus, Staphylococcus intermedius, Staphylococcus lugdunensis, Staphylococcus pasteuri, Staphylococcus saccharolyticus, Staphylococcus saprophyticus, Staphylococcus schleiferi schleiferi, Staphylococcus s. coagulans, Staphylococcus sciuri, Staphylococcus simulans, Staphylococcus warneri, Staphylococcus xylosus, Streptococcus agalactiae, Streptococcus canis, Streptococcus dysgalactiae dysgalactiae, Streptococcus dysgalactiae equisimilis, Streptococcus equi equi, Streptococcus equi zooepidemicus, Streptococcus iniae, Streptococcus porcinus, Streptococcus pyogenes, Streptococcus anginosus, Streptococcus constellatus constellatus, Streptococcus constellatus pharyngidis, Streptococcus intermedius, Streptococcus mitis, Streptococcus oralis, Streptococcus sanguinis, Streptococcus cristatus, Streptococcus gordonii, Streptococcus parasanguinis, Streptococcus salivarius, Streptococcus vestibularis, Streptococcus criceti, Streptococcus mutans, Streptococcus ratti, Streptococcus sobrinus, Streptococcus acidominimus, Streptococcus bovis, Streptococcus equinus, Streptococcus pneumoniae, Streptococcus suis, Vibrio alginolyticus, V, carchariae, Vibrio cholerae, C. cincinnatiensis, Vibrio damsela, Vibrio fluvialis, Vibrio fumissii, Vibrio hollisae, Vibrio metschnikovii, Vibrio mimicus, Vibrio par ahaemolyticus, Vibrio vulnificus, Yersinia pestis, Yersinia aldovae, Yersinia bercovieri, Yersinia enterocolitica, Yersinia frederiksenii, Yersinia intermedia, Yersinia kristensenii, Yersinia mollaretii, Yersinia pseudotuberculosis and Yersinia rohdei.

Alternatively, the molecules described herein may find use in treating a virus from a family selected from the group consisting of Astroviridae, Caliciviridae, Picornaviridae, Togaviridae, Flaviviridae, Caronaviridae, Paramyxviridae, Orthomyxoviridae, Bunyaviridae, Arenaviridae, Rhabdoviridae, Filoviridae, Reoviridae, Bornaviridae, Retroviridae, Poxviridae, Herpesviridae, Adenoviridae, Papovaviridae, Parvoviridae, Hepadnaviridae, (eg., a virus selected from the group consisting of a Coxsackie A-24 virus Adeno virus 11, Adeno virus 21, Coxsackie B virus, Borna Diease Virus, Respiratory syncytial virus, Parainfluenza virus, California encephalitis virus, human papilloma virus, varicella zoster virus, Colorado tick fever virus, Herpes Simplex Virus, vaccinia virus, parainfluenza virus 1, parainfluenza virus 2, parainfluenza virus 3, dengue virus, Ebola virus, Parvovirus B19 Coxsackie A-16 virus, HSV-1, hepatitis A virus, hepatitis B virus, hepatitis C virus, hepatitis D virus, hepatitis E virus, human immunodeficiency virus, Coxsackie B1-B5, Influenza viruses A, B or C, LaCross virus, Lassavirus, rubeola virus Coxsackie A or B virus, Echovirus, lymphocytic choriomeningitis virus, HSV-2, mumps virus, Respiratory Synytial Virus, Epstein-Barr Virus, Poliovirus Enterovirus, rabies virus, rubivirus, variola virus, WEE virus, Yellow fever virus and varicella zoster virus).

Alternatively, the molecules described herein may find use in treating a yeast or fungus. For example, a fungus or yeast that infects a host is selected from the group consisting of Aspergillus sp., Dermatophytes, Blastomyces dermatitidis, Candida sp., Histoplasma capsulatum, Sporothrix schenckii, Histoplasma capsulatum and Dematiaceous Fungi.

As used herein, the term “parasite” or “parasitological infection” shall be taken to mean an organism, whether unicellular or multicellular, other than a virus, bacterium, fungus or yeast that is capable of infecting another organism, for example a human. Examples of such parasites include, for example, a parasite selected from the group consisting of Ancylostoma ceylanicum, Ancylostoma duodenale, Ascaris lumbricoides, Balantidium coli, Blastocystis hominis, Clonorchis sinensis, Cyclospora cayetanensis, Dientamoeba fragilis, Diphyllobothrium latum, Dipylidium caninum, Encephalitozoon intestinalis, Entamoeba histolytica, Enterobius vermicularis, Fasciola hepatica, Enterobius vermicularis, Fasciola hepatica, Fasciolopsis buski, Giardia intestinalis (syn. Giardia lamblia), Heterophyes heterophyes, Hymenolepis diminuta, Hymenolepis nana, Isospora belli, Metagonimus yokogawai, Necator americanus, Opisthorchis felineus, Paragonimus westermani, Schistosoma haematobium, Schistosoma intercalatum, Schistosoma japonicum, Schistosoma mansoni, Taenia saginata, Trichuris trichiura, Babesia diver gens, Plasmodium falciparum, Plasmodium malariae, Plasmodium ovale, Plasmodium vivax, Leishmania braziliensis and Leishmania donovani.

In aspects, the molecules could be used generally to reduce biofilm formation or to disrupt already-formed biofilms. The molecules could also find use in down-regulating virulence genes, typically those associated with T3SS, and in reducing attachment of pathogens to tissue and/or surfaces. The treatment of wounds and treatment and/or prevention of infections in wounds using the molecules described herein is also contemplated.

In certain aspects, the treatment of specific enteric infections is contemplated. For example, Mycobacterium avium subspecies paratuberculosis is responsible for Johne's disease in cattle. The U.S. dairy industry has reported annual losses of $1.5 billion due to the disease and that 22% of the dairy herds in the U.S. are infected. It has a T3SS and would therefore expected to be treated and/or prevented through use of the molecules described herein.

In more general aspects, the molecules could be used as an alternative or adjunct to conventional antibiotic therapies to thereby reduce antibiotic use and mitigate the development of antibiotic resistance.

The molecules described herein can, in aspects, be administered for example, by parenteral, intravenous, subcutaneous, intradermal, intramuscular, intracranial, intraorbital, ophthalmic, intraventricular, intracapsular, intraspinal, intracisternal, intraperitoneal, intranasal, intrarectal, intravaginal, aerosol or oral administration. Typically, the compositions are administered orally or directly to the site of infection.

The molecules described herein may, in aspects, be administered in combination, concurrently or sequentially, with conventional treatments for infection, including antibiotics, for example. The molecules described herein may be formulated together with such conventional treatments when appropriate.

The molecules described herein may be used in any suitable amount, but are typically provided in doses comprising from about 1 to about 10000 ng/kg, such as from about 1 to about 1000, about 1 to about 500, about 10 to about 250, or about 50 to about 100 ng/kg, such as about 1, about 10, about 25, about 50, about 75, about 100, about 150, about 200, about 250, about 300, or about 500 ng/kg.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1: Postbiotic Metabolites Reduce Necrotic Enteritis Symptoms in Broilers Challenged with Clostridium perfringens

### Abstract

Necrotic enteritis caused by Clostridium perfringens is a major disease that can impact the broiler raised without antibiotic production market. Antibiotic alternatives are being developed for this growing market. Postbiotic metabolites are derived from the cell-free supernatant of probiotic fermentation and they interrupt cell-to-cell communication in pathogenic bacteria including Clostridium perfringens. The objective of this study was to determine if postbiotic metabolites could reduce the symptoms of necrotic enteritis in broilers that were orally challenged with Clostridium perfringens in the feed. Broilers were divided into 5 different treatment groups: (1) No Treatment Control (NTC); (2) Postbiotic metabolites 12 mg/kg BW (N-12); (3) Postbiotic metabolites 6 mg/kg BW (N-6); (4) Postbiotic metabolites 3 mg/kg BW (N-3); (5) salinomycin 0.042% in feed (SF). The birds were orally challenged with Clostridium perfringens on day 17 and lesion scores from 5 random birds per pen were assessed on day 21. Total and necrotic enteritis associated mortality was assessed daily while growth and performance were recorded on day 0, 17, 28 and 35. N-12 treatment significantly reduced the necrotic lesions assessed on day 21 compared to the NTC treatment. The N-12 treatment significantly reduced necrotic enteritis associated mortality between day 17-28 compared to the NTC treatment leading to an increase in the total pen weight harvested. Moreover, N-12, N-6, and N-3 treatments reduced the percentage of caked litter in the pens compared to both the NTC and SF treatments potentially leading to additional benefits to the cost of production.

### Introduction

Necrotic enteritis (NE) is a poultry disease that is caused by Clostridium perfringens. C. perfringens is a commensal bacterium that is found in the ceca of poultry and up to 37% of broilers can be affected by NE (Annett et al., 2002). Environmental conditions can cause this commensal bacterium to become pathogenic leading to lesions in the intestine and liver causing reduced feed conversion and increased mortality. However, the aetiology of why commensal C. perfringens transitions to a pathogenic bacterium is multifactorial and can be associated with changes in the dietary stresses, disruption of the microbiota, immune suppression and the presence of NetB⁺ strains (Moore 2016). Clinical symptoms are associated with necrotic lesions and high flock mortality, while sub-clinical symptoms are associated with poor feed conversions and growth performance. Both the clinical and sub-clinical symptoms can have a high economic impact on the industry due to the high mortality and poor performance. Previously, NE has been controlled by in-feed antibiotics but changes to these practices result in increased incidences of NE leaving poultry producers looking for alternative control methods.

Antibiotics are extensively used in the poultry industry and in 2014 there were 27 antibiotics that were registered in Canada for chickens, 15 were registered for coccidosis and 4 registered for NE (Diarra and Malouin, 2014). Some of the antibiotics registered for coccidiosis are also effective against Gram-positive bacteria including C. perfringens which indirectly increase the number of antibiotics registered for NE (Williams, 2005). Moreover, consumer demand and changes to governmental policy are impacting the use of antibiotics in the poultry industry and will impact enteric disease including coccidosis and NE (Cervantes, 2015). Live vaccines have been developed for coccidosis however, they can predispose the birds to NE outbreaks due to intestinal damage (Cervantes, 2015). Taken together, all of these factors influence the need to develop effective antibiotic alternatives for NE. Antibiotic alternatives that have been tested for NE include: vaccines, probiotics, prebiotics, organic acids, essential oils, botanicals and bismuth (Kulkarni et al., 2007; Caly et al., 2015; Timbermont et al., 2010; Diarra and Malouin, 2014; Stringfellow et al., 2009). Most of these antibiotic alternatives demonstrate strong in vitro efficacy however in vivo efficacy is dependent on limiting the growth and proliferation of C. perfringens which can lead to variability between trial replicates (Timbermont et al., 2010; Stringfellow et al., 2009). This variability is part of the problem with studying multifactorial pathogens such as C. perfringens. In addition, the virulence factors between strains can also be different making targeted vaccines difficult to develop (Kulkarni et al., 2007). An alternative approach in developing an effective antibiotic alternative is described herein, which is to target the bacterial communication system which regulates its virulence, leading to a product that is not dependent on the C. perfringens strain nor the population in the different compartments of the GI tract.

Postbiotic metabolites are specific metabolites from lactic acid bacteria that naturally interfere with bacterial cell-to-cell communication also known as quorum sensing. Quorum sensing regulates bacterial virulence factors including attachment, invasion, and toxin production and inhibiting quorum sensing using postbiotic metabolite can attenuate the expression of virulence factors. The objective of this research is to determine if a specific postbiotic metabolite composition, Nuvio, can reduce the symptoms of NE caused by an oral challenge of C. perfringens in broilers.

### Methods

Bird Placement. The birds in this study were sourced from a commercial hatchery and were housed at the Colorado Quality Research facility (CQR). Healthy male Cobb 500 were used in this study and were vaccinated for Mareks at the hatchery and upon arrival at CQR the birds were vaccinated for Newcastle and Infectious Bronchitis and were randomly divided into five treatments. At placement 24 birds were placed into each pen with 11 replicates per treatment. On day eight birds were culled or replaced as needed to have 22 birds per pen with the pen being the statistical unit. The treatments were assigned to blocks and randomized within blocks. All birds in this experiment were discarded on site. All data was collected by the Colorado Quality Research (CQR) staff. The protocol MS-18-1 was approved by the CQR IACUC committee.

Basal and Experimental Diets. The starter, grower, and basal diets were manufactured at the CQR feed mill using CQR formulated diets and described in Table 1. The feed was made and stored in bulk. The final experimental diet mixing, pelleting and crumbling was conducted at CQR using a 500-lb capacity vertical mixer (Seedburo, Golden Valley, Minn.), a 4000-lb capacity vertical mixer (Prater, Bolingbrook, Ill.) and/or a 14,000-lb horizontal mixer (H&S Manufacturing, Marshfield, Wis.) and a California pellet mill (CPM Co., Crawfordsville, Ind.). The feed was stored in 50-lb feed sacks until needed.

TABLE 1
Feed schedule for three phase diets during the trial.

| Diet | Form | Period | ~Lbs Feed to Mix per treatment |
|---|---|---|---|
| Starter | Crumble | Day 0-17 | ~550 |
| Grower | Pellet | Day 17-28 | ~815 |
| Finisher | Pellet | Day 28-35 | ~650 |

Growth Conditions. The birds were housed in an environmentally controlled facility with concrete floor pens (˜4′×4′ minus 2.25 sq ft. for the feeder. The bird density at D0 was ˜0.573 ft²/bird, D7 ˜0.625 ft²/bird, and D21 ˜0.809 ft²/bird. The birds were housed with wood shavings and have water and food ad libitum, supplemental lighting will be used as indicated in Table 2.

TABLE 2
Lighting regime and intensity used during the trial.

| | Approximate hours of | |
|---|---|---|
| Approximate bird age | continuous light per | ~Light Intensity |
| (days) | 24 hour period | (Foot candles) |
| 0-5 | 24 | 1.0-1.3 |
| 5-11 | 12 | 1.0-1.3 |
| 11-19 | 12 | 0.2-0.3 |
| 19-30 | 16 | 0.2-0.3 |
| 30-35 | 18 | 0.2-0.3 |

Experimental Treatments and Trial Observations. The experimental treatments are described in Table 3. The untreated control NTC birds were not given medication in the feed or water. The treatments N-12, N-6 and N-3 were administered Nuvio (MicroSintesis Inc, Charlottetown, PE) in the drinking water from day 12 to 28 at 12 mg/kg BW/day, 6 mg/kg BW/day and 3 mg/kg BW/day respectively.

TABLE 3
Experimental design.

| | Treatment | Test Article Inclusion | | | No. of | No. | No. of |
|---|---|---|---|---|---|---|---|
| | Description/ | Level | | | birds/ | of | birds/ |
| Trt. | Duration | Nuvio | Salinomycin | Administration | pen¹ | Pens | treatment¹ |
| NTC | Untreated | — | — | — | 24(22) | 11 | 264 (242) |
| | Control | | | | | | |
| N-12 | Nuvio 1x D12- | 12 | — | Water | 24(22) | 11 | 264 (242) |
| | 28 | mg/kg | | | | | |
| | | BW/day | | | | | |
| N-6 | Nuvio 1/2x | 6 mg/kg | — | Water | 24(22) | 11 | 264 (242) |
| | D12-28 | BW/day | | | | | |
| N-3 | Nuvio 1/4x | 3 mg/kg | — | Water | 24(22) | 11 | 264 (242) |
| | D12-28 | BW/day | | | | | |
| SF | Salinomycin | — | 0.042% | Feed | 24(22) | 11 | 264 (242) |

¹Number of birds placed at the start of the trial (number of birds after re-count on day 8)

The product was made fresh daily and administered in the water using a liquid medicator (Farmer Boy Ag, Myerstown, Pa.). A different stock solution was prepared for each treatment and the stock solutions were weighed each day to determine the amount of product consumed. The stock solutions were adjusted accordingly to ensure that the correct dose has been administered based on water consumption. The antibiotic control SF birds were administered Salinomycin (Sacox 60, Phibro Animal Health Corp., Teaneck, N.J.) in the feed at 0.042% for the whole experimental period. The test facility, pens and birds were observed at least twice daily for general flock condition, lighting, water, feed, ventilation and unanticipated events. If abnormal conditions or abnormal behavior is noted at any of the twice-daily observations they were documented and added to the study records. The maximum and minimum temperatures were recorded daily. The dead birds were collected and counted each day from each pen. The mortality was recorded on pen sheet and the cause of death was recorded. The pen mortality and the NE-associated mortality were calculated based on total mortality and as a percentage. The percentages were calculated based on the number of re-count birds on day 8. The birds were weighed on day 0, 17, 28, and 35 and the average daily gain was calculated for each of these time periods. The final pen weight was also measured. The amount of feed consumed was calculated from the starter feed (D0-17), the grower feed (D18-28), finisher feed (D29-35), and over the whole period (D0-35). The feed efficiency and adjusted feed efficiency were calculated to compare different growth periods. On day 21, 5 birds from each pen were randomly selected using a first caught method. The birds were sacrificed and evaluated for intestinal lesions to score the severity of the lesion scores using the scale in Table 4.

TABLE 4
Scale used to evaluate lesion scores.

| Score | Description |
|---|---|
| 0—Normal | No NE lesions, small intestine has normal elasticity (rolls |
| | back to normal position after being opened) |
| 1—Mild | Small intestinal wall was thin and flaccid (remains flat |
| | when opened and does not roll back into normal position |
| | after being opened); excess mucus covering mucous |
| | membrane |
| 2—Moderate | Noticeable reddening and swelling of the intestinal wall; |
| | minor ulceration and necrosis of the intestine membrane; |
| | excess mucus |
| 3—Severe | Extensive area(s) of necrosis and ulceration of the small |
| | intestinal membrane; significant hemorrhage; layer of fibrin |
| | and necrotic debris on the mucous membrane (Turkish |
| | towel appearance) |
| 4—Dead or | A bird that would likely die within 24 hours and has NE |
| moribund | lesion score of 2 or more; or birds that died due to NE |

Preparation of C. perfringens Inoculum and Administration. The C. perfringens culture was obtained from Microbial Research Inc. The C. perfringens (CL-15, Type-A, a and (32 toxins) was administration via feed. CL-15 is a field strain of C. perfringens from a broiler outbreak in Colorado. The culture was grown for ˜5 hours at 37° C. in fluid thioglycolate medium containing starch. The C. perfringens CL-15 culture was mixed with feed on study day 17. Feed from each pen's feeder was removed from the birds for 4-8 hours. For each pen 2.5 ml/bird of the broth culture was mixed with 25 g of feed per bird in the feeder tray. The feed was consumed within 1-2 hours of administration.

Evaluation of Litter Score. The litter from each pen was evaluated on day 28 of the trial. The litter was evaluated based on percentage of caked litter in the pen. The percentages were grouped from 0-19, 20-39, 40-59, 60-79, and 80-100%. The mid-value from the percentage range was used to calculate the average pen litter score.

### Results

### Mortality and Necrotic Enteritis Associated Mortality

The bird mortality was collected throughout the trial and the cause of death was recorded. The trial was set-up into three periods based on diet: D0-17 starter (pre-challenge), D17-28 grower (post-challenge), and D28-35 finisher (post-challenge). The data in Table 5 indicates the NE-associated and total mortality during each of these periods. There was no NE associated mortality during the starter period, the mortality issues during this period were mostly due to sudden death syndrome, muscular-skeletal issues, and bacterial infections. The mortality during this period ranged from 0.41-3.7% for all treatments.

TABLE 5
Total mortality and necrotic enteritis associated mortality during different growth periods.

| | Day 0-17 | | Day 17-28 | | Day 28-35 | | Day 0-35 | |
|---|---|---|---|---|---|---|---|---|
| | NE | Total | NE | Total | NE | Total | NE | Total |
| Trt. | Mortality | Mortality | Mortality | Mortality | Mortality | Mortality | Mortality* | Mortality* |
| NTC | N/A | 7 | 37 | 43 | 4 | 4 | 41 | 54 |
| | | (2.9%) | (15.3%) | (17.8%) | (1.7%) | (1.7%) | (16.9%) | (22.3%) |
| N-12 | N/A | 9 | 24 | 28 | 3 | 4 | 27 | 41 |
| | | (3.7%) | (9.9%) | (11.6%) | (1.2%) | (1.7%) | (11.2%) | (16.9%) |
| | | | p = | p = | | | p = | p = 0.012 |
| | | | 0.0001 | 0.0024 | | | 0.0067 | |
| N-6 | N/A | 4 | 36 | 38 | 3 | 5 | 39 | 47 |
| | | (1.7%) | (14.9%) | (15.7%) | (1.2%) | (2.1%) | (16.1%) | (19.4%) |
| | | | p = | p = | | | p = 0.441 | p = 0.111 |
| | | | 0.519 | 0.0959 | | | | |
| N-3 | N/A | 1 | 49 | 52 | 0 | 2 | 49 | 55 |
| | | (0.41%) | (20.3%) | (21.5%) | (0%) | (0.8%) | (20.3%) | (22.7%) |
| | | | p = | p = | | | p = 0.0004 | p = 0.831 |
| | | | 0.0455 | 0.0306 | | | | |
| SF | N/A | 5 | 4 | 5 | 1 | 3 | 5 | 13 |
| | | (2.1%) | (1.7%) | (2.1%) | (0.4%) | (1.2%) | (2.1%) | (5.4%) |
| | | | p = | p = | | | p = | p = |
| | | | 0.0001 | 0.0001 | | | 0.0001 | 0.0001 |

Parenthesis indicate the mortality expressed as a percentage of birds per treatment on day 8 after re-count
*Significance level based on the ranked pen comparison of necrotic enteritis associated mortality compared to the control treatment

The birds were challenged with C. perfringens in the feed on day 17 of the trial. After the oral challenge, there was an increase in the NE-associated mortality in all treatment groups. The SF group had the least mortality at 1.7%, followed by the N-12 group with 9.9%. Both of these treatment groups were statistically different (P=0.0001) compared to the NTC birds when the mortality compared based on differences in pen severity. The other two groups N-6 and N-3 had 14.9% and 20.3% NE associated mortality but were not statistically different than the control group with 15.3%. The total mortality during this period showed a similar trend indicating that the other mortality was similar between all groups. The pen ranked comparison evaluates the differences based on severity since not all pens in the control group displayed the same level clinical symptoms although they were given the same dose of C. perfringens. This type of analysis can be used to evaluate how well a treatment can perform based on the level of challenge.

The mortality during the finisher period decreased for all treatment groups. The NE associated mortality during this period ranged from 0-1.7% and the total mortality ranged from 0.8-2.1%. The data in Table 5 indicates that the majority of the mortality was associated with NE and was most prevalent during the grower phase. Over the 35 day period the lowest NE associated mortality was in the SF group with 2.1% (P=0.0001) followed by the N-12 group with 11.2% mortality (P=0.0067). The N-6 group was not statistically different than the negative control indicating that the N-12 was the lowest effective dose to reduce NE associated mortality. Over the whole experimental period there were 41 birds that died due to NE in the negative control while only 27 died in the N-12 group this is a 34% reduction in total NE-associated mortality.

### Intestinal Lesion Scoring

The birds were evaluated on day 21 for necrotic lesions in the small intestine. Five birds from each pen were randomly caught and euthanized. A post-mortem was performed and the small intestine was opened to score the severity of the NE-associated lesions using the criteria described in Table 4. The lesion scores from the 5 birds were averaged to calculate the pen lesion score. The treatment averages are indicated in Table 6 and the statistical significance was calculated based on a rank pen comparison. The average lesion score in the NTC group was 1.62 and 1.38 in the N-12 which is a 14.8% reduction in the lesions scores. These two treatments were statistically different P=0.039. However, the N-6 and N-3 were not statistically different than the NTC group based on a pen ranked difference. The SF treatment statistically reduced the average lesion score to 0.80 which is a 50.6% reduction compared the NTC birds. Since the other two treatments N-6 and N-3 did not show a statistical difference compared to the NTC, the N-12 was the lowest effective dose to reduce necrotic lesions with this C. perfringens CL-15 strain.

TABLE 6
Evaluation of necrotic enteritis lesion scores on day 21.

| Treatment | Average lesion score | Significance level* |
|---|---|---|
| NTC | 1.62 | — |
| N-12 | 1.38 | p = 0.039 |
| N-6 | 1.64 | p = 0.875 |
| N-3 | 1.93 | p = 0.094 |
| SF | 0.80 | p = 0.0006 |

*Comparison of the pen ranked difference

### Growth Performance and Feed Efficiency

The data in Table 7 indicate that neither Nuvio nor salinomycin statistically improved the weight gain or the average daily weight gain during any of the periods. However, there was a numerical improvement in the weight gain and average daily gain during the grower post-challenge period (D17-28) for both N-12 and N-6 compared to the negative control. The N-12 group grew an extra 5 grams per bird (+0.53%) and the N-6 grew 7 gram per bird (+0.75%) compared to the NTC. Conversely, the SF group grew 2 grams (−0.21%) less than the NTC birds during the same period.

TABLE 7
Average weight gain per bird and
average daily gain per bird during different growth periods

| | Period | | | | | | | | | |
|---|---|---|---|---|---|---|---|---|---|---|
| | D 0-17 | | D 0-28 | | D 0-35 | | D 17-28 | | D 28-35 | |
| | | Avg. | | Avg. | | Avg. | | Avg. | | Avg. |
| | Weight | daily | Weight | daily | Weight | daily | Weight | daily | Weight | daily |
| | gain | gain | gain | gain | gain | gain | gain | gain | gain | gain |
| Treatment | (kg) | (g/d) | (kg) | (g/d) | (kg) | (g/d) | (kg) | (g/d) | (kg) | (g/d) |
| NTC | 0.476 | 28.0 | 1.406 | 50.2 | 2.072 | 59.2 | 0.930 | 84.5 | 0.666 | 95.1 |
| N-12 | 0.474 | 27.9 | 1.409 | 50.3 | 2.086 | 59.6 | 0.935 | 85.0 | 0.677 | 96.7 |
| N-6 | 0.473 | 27.8 | 1.409 | 50.3 | 2.077 | 59.3 | 0.937 | 85.1 | 0.668 | 95.4 |
| N-3 | 0.478 | 28.1 | 1.398 | 49.9 | 2.063 | 58.9 | 0.920 | 83.6 | 0.665 | 95.0 |
| SF | 0.479 | 28.1 | 1.407 | 50.3 | 2.087 | 59.6 | 0.928 | 84.4 | 0.680 | 97.1 |

No statistical difference

The feed conversion ratio (FCR) and adjusted FCR is presented in Table 8. The data indicated that Nuvio treatments and salinomycin did not statistically improve these growth parameters. However, during the challenge period (D17-28) there was a 14.6% improvement in the FCR in the N-12 compared to the negative control, an 11.5% improvement in the FCR with N-6, a 0.5% improvement with the N-3 treatment. There appears to be a dose response with the improvement of the FCR during the challenge period and the amount of Nuvio administered. A similar trend was observed for the adjusted FCR for both the N-6 and N-12 doses. Moreover both the FCR and adjusted FCR were better than the negative control for the N-6 and N-12 dose over the whole 0-35 day period albeit at a lower percent difference. The FCR for the N-12 dose had a 2.4% improvement and 0.82% for the N-6 dose and similarly, the adjusted FCR had a 0.34% improvement for the N-12 dose and 0.14% for the N-6 dose. The improvement in the feed conversion during the challenge period suggests that Nuvio may allow the birds to digest food more efficiently despite having necrotic lesions. This is even more pronounced with the N-6 dose where there was no statistical difference in the lesion score 1.62 compared to 1.64 or in NE mortality 46 to 39 but there was a 11.5% improvement in the FCR compared to the NTC. This improvement may be relevant in sub-clinical NE cases where there is low mortality but a large impact on feed efficiency.

TABLE 8
Feed-to-gain and adjusted feed-to-gain ratios during different growth periods.

| | Period | | | | | | | | | |
|---|---|---|---|---|---|---|---|---|---|---|
| | D 0-17 | | D 0-28 | | D 0-35 | | D 17-28 | | D 28-35 | |
| | | Adj. | | Adj. | | Adj. | | Adj. | | Adj. |
| Treatment | F:G | F:G | F:G | F:G | F:G | F:G | F:G | F:G | F:G | F:G |
| NTC | 1.331 | 1.296 | 1.838 | 1.395 | 1.812 | 1.474 | 2.610 | 1.471 | 1.767 | 1.700 |
| N-12 | 1.334 | 1.298 | 1.774 | 1.388 | 1.769 | 1.469 | 2.229 | 1.449 | 1.775 | 1.697 |
| N-6 | 1.333 | 1.302 | 1.791 | 1.390 | 1.797 | 1.472 | 2.309 | 1.451 | 1.820 | 1.705 |
| N-3 | 1.338 | 1.302 | 1.874 | 1.407 | 1.823 | 1.478 | 2.596 | 1.486 | 1.720 | 1.684 |
| SF | 1.320 | 1.287 | 1.656 | 1.368 | 1.666 | 1.448 | 1.947 | 1.421 | 1.691 | 1.654 |

### No Statistical Difference

Although there was no statistical difference in the any of the bird weights for any treatment, there was a statistical difference between the final pen weights since the final pen weights take into consideration the differences in the bird mortality. The data in Table 9 indicates that the heaviest pens were associated with the SF treatment with an average pen weight of 34.59 kg. The N-12 and N-6 doses were statistically different from the other three treatments but there was no statistical difference between these two doses, 30.87 and 30.14 kg respectively. The N-3 and the NTC were not statistically different, 29.22 and 29.26 kg respectively. It is evident that the mortality had a large impact on the final pen weight, leading to a 5.5% increase in the harvest weight for N-12 compared to the NTC and 3.0% for the N-6 dose. As expected the SF had an 18.2% increase in the harvest weight due to the large mortality reduction.

TABLE 9
Live pen weights at day 35.

| Treatment | Average pen weight (kg) |
|---|---|
| NTC | 29.26c |
| N-12 | 30.87b |
| N-6 | 30.14b |
| N-3 | 29.22c |
| SF | 34.59a |

### Letters Indicate Statistical Significance LSD p <0.05 One-Tail Test

### Evaluation of Litter

The litter from each pen was evaluated on day 28 of the trial. The percentage of caked litter was evaluated using a percentage range in 20% increments. Caked litter is an indicator of litter moisture. The SF treatment had an average percentage of 79% and was statistically higher than the NTC 72%. The Nuvio treatments had a drier litter with the N-12 having the driest litter 59%, N-6 63% and N-3 65%. The N-12 and N-6 treatments were statistically lower than the NTC treatment. This indicates that birds that received Nuvio had drier litter compared to the NTC and SF treatments.

### Discussion

Nuvio contains postbiotic metabolites which are metabolites that are produced during the fermentation of probiotic bacterium such as Enterococcus faecium. The broilers were grown in small pens and orally challenged with C. perfringens in the feed. The symptoms of necrotic enteritis were indicated by mortality, necrotic lesions of the small intestine and feed efficiency. Nuvio at three different doses was compared to salinomycin, an ionophore that is commercially used to prevent necrotic enteritis as well as an NTC. The data indicated that N-12 was the minimum effective dose to reduce mortality and necrotic lesions. This dose was able to reduce the necrotic enteritis-associated mortality by 34% compared to the NTC. The reduction in mortality was statistically different and indicates that Nuvio is an effective antibiotic alternative.

Reduction in mortality is important when considering the economic impact to the producer since NE usually affects older birds with higher feed inputs. Unfortunately, NE-associated mortality is not recorded either because a low virulent strain was used or experimental design did not allow for statistical comparisons (Stringfellow et al., 2009; Timbermont et al., 2010). Conversely, the assessment of necrotic lesions is often part of broiler studies. In this study the N-12 dose reduced the lesion score from 1.62 to 1.38 which is a 14.8% reduction. Often the lesion score is positively correlated with the C. perfringens CFU/g (Timbermont et al., 2009; McReynolds et al., 2009) however, there are often large differences in the C. perfringens CFU/g and the lesion score, for example, the positive control with the same strain yielded a lesion score of 1.33 and 1.28 while the CFU/g was 2630 and 61659 respectively (McReynolds et al., 2009). Moreover, comparing two strains from two different studies one positive control had a lesion score of 2.34 with a C. perfringens CFU/g of 1737 while the other positive controls from the other study had lesion from different trials of 1.33, 1.28 and 2.15 and C. perfringens CFU/g values of 2630, 61659, and 91201 respectively (Timbermont et al., 2009; McReynolds et al., 2009). This large variation in C. perfringens population and small differences in lesion scores suggests that the virulence of the pathogen is important as well as the environmental factors that influence the virulence.

The reduction in lesion score allowed the birds to improve their FCR, the birds receiving N-12 had a 14.6% improvement compared to the control during the challenge period. Although the data was not statistically different than the control this positive improvement suggests that Nuvio can improve the feed efficiency while the birds are challenged with and recovering from C. perfringens. Despite the fact, there were no differences in the final bird weight from any other the treatments the N-12 was able to increase the final pen weight by 5.5% which can have a dramatic improvement to producers' profit at the large production scale. Taken together the data indicates that N-12 dose could be effective to treat a medium to high virulent strain of C. perfringens since its mode of action is dependent on attenuating the virulence of the pathogen rather than inhibiting its growth.

The N-6 dose was not able to reduce the necrotic enteritis associated mortality or the lesion scores but there was a numerical improvement in the FCR during the challenge period as well as a statistical improvement in the final pen weight. This data indicates that this dose may be effective at reducing the sub-clinical signs of necrotic enteritis which are associated with growth and performance. This dose was able to improve the FCR by 11.5% and the final pen weight by 3%. The sub-clinical cases of necrotic enteritis occur frequently in the broiler industry and can have a large impact on the producers profits due to lower final bird weights and feed wastage (M'Sadeq et al., 2015). This dose maybe effective at treating sub-clinical necrotic enteritis or improving flock health in non-challenge situations. Taken together this data suggests that the cell-free supernatant from Lactobacillus cultures can positively impact the microbiome and improve the microbiome barrier function resulting in improved livestock health and performance.

There are many factors that can effect litter dryness including flock density, ventilation, depth of litter, diet, and bacterial infections (Williams, 2005; Cervantes, 2015). The data in Table 10 indicates that birds receiving Nuvio had drier litter scores than the NTC and SF treated birds. Assuming that the flock density, ventilation, litter depth and diet were the same between all treatments it is likely that Nuvio was impacting an enteric bacterial infection or improving the microbiome of the bird resulting in drier droppings. The drier litter may reduce future infections from oral ingestion of the litter since up to 6.3% of the birds feed can come from the litter (Malone et al., 1983) and wet litter can be a source for pathogen growth from droppings. Principle component analysis indicated that litter moisture is the main factor influencing the microbial population in the litter (Lovanh et al., 2007). The population of bacteria found in the litter were also similar to the populations that are found in the ileum and cecae which can be dominated by Staphylococcus spp (Lovanh et al., 2007; Pedroso et al., 2013). Wet litter can also increase the volatilization of ammonia and the amount of ammonia producing bacteria (Rothrock et al., 2008). The increased ammonia in the litter and atmosphere can also lead to increased hockburn and foot pad dermatitis (Shepherd and Fairchild, 2010) as well as respiratory ailments for the birds and workers (Ritz et al., 2004). The positive impact that Nuvio has on the litter moisture suggests it is a result of changes to the microbiome and the potential use of Nuvio for improving flock health by potentially reducing ammonia, improving bird welfare and reducing the spread of pathogenic bacteria.

TABLE 10
Average percent caked litter per treatment on day 28.

| Treatment | Value (%) | p-value |
|---|---|---|
| NTC | 72 | N/A |
| N-12 | 59 | 0.0019 |
| N-6 | 63 | 0.0162 |
| N-3 | 65 | 0.1039 |
| SF | 79 | 0.0379 |

The data presented in this paper indicates that Nuvio can be used as an effective antibiotic alternative at reducing NE. There are many positive attributes to Nuvio including the reduction in mortality and lesion scores and improvements to the final pen weights and FCR. The unique mode of action for Nuvio allows it to be differentiated from other antibiotic alternatives that rely on either suppressing bacterial growth or modifying the local environment to suppress growth. NE is a multifactorial disease and the virulence of C. perfringens is dependent on the strain and the environmental factors that trigger the expression of the virulence factors. However, Nuvio can attenuate the virulence of many pathogenic bacteria including C. perfringens by interfering with quorum sensing which is a main regulator of bacterial virulence.

### REFERENCES

Annett, C. B., J. R. Viste, M. Chirino-Trejo, H. L. Classen, D. M. Middleton, and E. Simko. 2002. Necrotic enteritis: effect of barley, wheat and corn diets on proliferation of Clostridium perfringens type A. Avian Pathol. 31:599-602.
Bayoumi, M. A., and M. W. Griffiths. 2010 Probiotics down-regulate genes in Salmonella enterica Serovar Typhimurium pathogenicity island 1 and 2. J. Food Prot. 73: 452-460.
Bayoumi, M. A., and M. W. Griffiths. 2012 In vitro inhibition of expression of virulence genes responsible for colonization and systemic spread of enteric pathogens using Bifidobacterium bifidum secreted molecules. Int. J. Food Microbiol. 156:255-263.
Caly, D. L., R. D'Inca, E. Auclair, and D. Drider. 2015. Alternatives to antibiotics to prevent necrotic enteritis in broiler chickens: A microbiologist's perspective. Front. Microbiol. 6: 1336.
Cervantes, H. M. 2015 Antibiotic-free poultry production: Is it sustainable? J. Appl. Poult. Res. 24:91-97.
Diarra, M. S., and F. Malouin. 2014. Antibiotics in Canadian poultry productions and anticipated alternatives. Front Microbiol. 5:282
Kareem, K. Y., T. C. Loh, H. L. Foo, S. A. Asmara, and H. Akit. 2017. Influence of postbiotic RG14 and inulin combination on cecal microbiota, organic acid concentration, and cytokine expression in broiler chickens. Poult. Sci. 96:966-975.
Kim, Y., J. W. Lee, S-G Kang, S. and M. W. Griffiths. 2012. Bifidobacterium spp. influences the production of autoinducer-2 and biofilm formation by Escherichia coli O157:H7. Anaerobe 18:539-545.
Kulkarni, R. R., V. R. Parreirra, S. Sharif, and J. F. Prescott. 2007. Immunization of broiler chickens against Clostridium perfringens-induced necrotic enteritis. Clin. Vaccine Immunol. 14:1070-1077.
Loh, T. C., T. V. Thu, H. L. Foo, and M. H. Bejo. 2013. Effects of different levels of metabolite combination produced by Lactobacillus plantarum on growth performance, diarrhea, gut environment and digestibility of postweaning piglets. J. Appl. Anim. Res. 41: 200-207.
Lovanh, N., K. L. Cook, M. J. Rothrock, D. M. Miles, and K. Sistani. 2007. Spatial shifts in microbial population structure within poultry litter associated with physiochemical properties. Poult. Sci. 86:1840-1849.
Malone, G. W., and G. W. Chaloupka 1983. Influence of litter type and size on broiler performance. 1. Factors affecting litter consumption. Poult. Sci. 62:1741-1746.
McReynolds, J., C. Waneck, J. Byrd, K. Genovese, S. Duke, and D. Nisbet. 2009. Efficacy of multistrain direct-fed microbial and phytogenic products in reducing necrotic enteritis in commercial broilers. Poult. Sci. 88:2075-2080.
Medellin-Pena, M. J., H. Wang, R. Johnson, S. Anand, and M. W. Griffiths. 2007. Probiotics affect virulence-related gene expression in Escherichia coli O157:H7. Appl. Environ. Microbiol. 73:4259-4267.
Medellin-Pena, M. J., and M. W. Griffiths. (2009) Effect of molecules secreted by Lactobacillus acidophilus strain LA5 on Escherichia coli O157:H7. Appl. Environ. Microbiol. 75:1165-1172.
Moore, R. J. 2016. Necrotic enteritis predisposing factors in broiler chickens. Avian Pathol. 45: 275-281.
M'Sadeq, S. A., S. Wu, R. A. Swick, and M. Choct. 2015. Towards the control of necrotic enteritis in broiler chickens with in-feed antibiotics phasing-out worldwide. Animal Nutrition 1: 1-11.
Mundi, A., V. Delcenserie, M. Amiri-Jami, S. Moorhead, and M. W. Griffiths. 2013. Cell-free preparations of Lactobacillus acidophilus strain La-5 and Bifidobacterium longum strain NCC2705 affect virulence gene expression in Campylobacterjejuni. J. Food Prot. 76:1740-1746.
Nordeste, R., A. Tessema, S. Sharma, Z. Kovač, C. Wang, R. Morales, and M. W. Griffiths. 2017. Molecules produced by probiotics prevent enteric colibacillosis in pigs. BMC Vet. Res. 13:335.
Pedroso, A. A., A. L. Hurley-Bacon, A. S. Zedek, T. W. Kwan, A. P. O. Jordan, G. Avellaneda, C. L. Hofacre, B. B. Oakley, S. R. Collett, J. J. Maurer, and M. D. Lee. 2013. Can probiotics improve the environmental microbiome and resistome of commercial poultry production? Int. J. Environ. Res. Public Health 10: 4534-4559.
Ritz, C. W., B. D. Fairchild, and M. P. Lacy. 2004. Implications of ammonia production and emissions from commercial poultry facilities: a review. J. Appl. Poult. Res. 13:684-692
Rothrock Jr., M. J., K. L. Cook, N. Lovanh, J. G. Warren, and K. Sistani. 2008. Development of a quantitative real-time polymerase chain reaction assay to target a novel group of ammonia-producing bacteria found in poultry litter. Poult Sci. 87:1058-1067.
Shepherd, E. M., and B. D. Fairchild. 2010. Footpad dermatitis in poultry. Poult. Sci. 89:2043-2051.
Stringfellow, K., J. McReynolds, J. Lee, J. Byrd, D. Nisbet, and Farnell, M. 2009. Effect of bismuth citrate, lactose, and organic acid on necrotic enteritis in broilers. Poult. Sci. 88:2280-2284.
Timbermont, L., A. Lanckriet, J. Dewulf, N. Nollet, K. Schwarzer, F. Haesebrouck, R. Ducatelle, and F. Van Immerseel. 2010. Control of Clostridium perfringens-induced necrotic enteritis in broilers by targeted-releaseed butyric acid, fatty acids and essential oils. Avian Pathol. 39:2, 117-121.
Troll, M. L. 2014. Investigating the anti-virulent activity of probiotic bioactives on Clostridium perfringens. M.Sc. Thesis University of Guelph.
Williams, R. B. 2005. Intercurrent coccidiosis and necrotic enteritis of chickens: rational, integrated disease management by maintenance of gut integrity. Avian Pathol. 34:159-180.
Yun, B., S. Oh, and M. W. Griffiths. 2014. Lactobacillus acidophilus modulates the virulence of Clostridium difficile. J. Dairy Sci. 97:4745-4758.
Zeinhom, M., A. M. Tellez, V. Delcenserie, A. M. El-Kholy, S. H. El-Shinawy, and M. W. Griffiths. 2012. Yogurt containing bioactive molecules produced by Lactobacillus acidophilus La-5 exerts a protective effect against enterohemorrhagic Escherichia coli in mice. J. Food Prot. 75:1796-1805.

### Example 2: A Pilot Study to Evaluate Safety of Postbiotic Metabolites from Enterococcus faecium Alone and in Combination with Probiotics in Dogs

### Abstract

This study evaluated the safety and dose tolerance of postbiotic metabolites from Enterococcus faecium and their combination with live cells of the microorganism (probiotics) when administered to Beagles. The study had a masked, randomized, controlled, parallel design. Twenty healthy Beagles were randomly allocated to five groups of four dogs each: control group (T0), three groups receiving 1 ×, 3×, and 5× the intended dose of postbiotic metabolites (T1/T3/T5, respectively) and a group receiving 5× dose with live cells (T5c). The safety assessment included physical and clinical examinations. Abnormal clinical signs included vomiting, blepharospasm, conjunctival hyperemia, epiphora, muco-purulent discharge in eyes and minor proteinuria but in all cases these symptoms were mild, transient, did not require veterinary intervention, and were not associated with the test article. There were no dose group effects, toxicological effects or serious adverse advents. Postbiotic metabolites (alone or in combination with probiotics) was safe and well-tolerated by dogs in the study.

### Introduction

Probiotics have been used in the nutrition of domestic animals for a long time, as they maintain or stabilise the composition and metabolic activity of the canine intestinal flora and modify immune function in dogs (1-3). Recent advances in nutrition have led to the emergence of postbiotic metabolites, which are specific metabolites produced during a proprietary fermentation process by selected microbial strains. They have been shown to interrupt cell-to-cell communication in pathogenic bacteria, resulting in a suppression of virulence. Postbiotic metabolites downregulate virulence genes in a wide variety of bacteria and exert an immunomodulatory effect on the host (4-6).

In some instances probiotic microorganisms can cause episodes of infection like fungemia, sepsis and bacteremia particularly in vulnerable and immunocompromised patients (12). This study was designed to determine the safety and dose tolerance of proprietary bacterial peptides, termed postbiotic metabolites and their combination with the probiotics Enterococcus faecium, in healthy, adult Beagle dogs.

### Methods

The study had a masked, randomized, controlled, parallel design. The study was comprised of five groups of four dogs each, they were housed individually according to standard facility practices. All dogs received certified Canine LabDiet®. The food met or exceeded the latest National Research Council requirements for energy, protein, vitamins, and minerals for the species and age class. Twenty (20) healthy Beagle dogs were randomly allocated to five groups (T0, T1, T3, T5, and T5c) of four dogs each. Group T0 was the control, groups T1, T3, T5 received postbiotic metabolites at doses of 1× (132.13 mg/kg), 3× (132.13 mg/kg), 5× (132.13 mg/kg), respectively, and group T5c received a 5× (132.13 mg/kg) dose of postbiotic metabolites together with live cells of Enterococcus faecium at 40 billion CFU per day. Dogs were dosed once daily as described in Table 11. The test facility complied with all regulations governing the care and use of laboratory animals. Procedures were designed to avoid or minimize discomfort, distress and pain to dogs in accordance with the principles of the Ontario Animals for Research Act (RSO 1990, Chapter A. 22); the U.S. Animal Welfare Act and Amendments (7USC and amendments); and the guidelines of Canadian Council on Animal Care.

TABLE 11
Distribution of groups and dose amongst dogs.

| Group | N | Route | Dose | Duration |
|---|---|---|---|---|
| T0 | 4 | Oral | Control | 28 days |
| T1 | 4 | Oral | 1× (12.13 mg/kg) | 28 days |
| T3 | 4 | Oral | 3× (396.39 mg/kg) | 28 days |
| T5 | 4 | Oral | 5× (660.65 mg/kg) | 28 days |
| T5c | 4 | Oral | 5× (660.65 mg/kg)^{a} | 28 days |

^{a}In combination with Lactobacillus cell culture at 40 billion CFUs per day.

Test facility personnel conducting clinical observations, laboratory analyses, or procedures that could influence study variables were blinded to the identity of the treatment administered. No un-blinding of blinded personnel occurred before the completion of the in-life phase of the study, including completion of all laboratory analyses. On Day −1, dogs selected to enter the dosing period were ranked by study animal ID in ascending order and randomly assigned to one of five dose groups using Microsoft EXCEL® (Microsoft Corporation, Redmond, Wash.). Age of the dogs ranged from 646 to 819 days.

Inclusion criteria included good physiological health, intact male Beagles older than six months (182 days) of age at the start of dosing, had no clinically significant health abnormalities based on pre-study physical examination, clinical pathology (hematology and serum chemistry) and urinalysis and were amenable to the study procedures. Dogs were excluded from the study if they did not meet inclusion criteria, they were inappetant; they had evidence of pre-study complicating disease that may interfere with or prevent the evaluations and analyses used in this study; or they had been treated with any medications in the 28 days prior to Day 0 that may influence study endpoints.

Dogs were dosed once daily from days 0 to 27. Dosing on any given day was performed prior to feeding. From days 0 to 6, dogs were dosed with test article packed in gelatin capsules. Due to a high number of vomitions, the test article preparation and dosing procedure were amended. On study days 7 to 27 dogs were dosed with a liquid formulation that was prepared daily using 1:1 ratio of test article and natural spring water.

Blood was collected from each dog for clinical chemistry and hematology analyses during acclimation (day −7), and treatment (days 14 and 28). Urinalysis was also performed on each dog during acclimation (day −7), and at days 14 and 27 of treatment. Clinical observations were performed at least once daily and included observations of eyes, mucous membranes, respiration, fecal consistency score, fecal appearance and mentation score. Each dog received a complete veterinary examination during acclimation (day −7), and at days 14 and 28 of treatment. Each dog's body weight was measured using a calibrated scale to the nearest 0.1 kg twice during acclimation on days −6 and −1 and during treatment (days 6, 13, 20, and 28). The weight of food offered and not eaten was measured daily beginning on Day −7 and continued until the completion of the study. Adverse events and serious adverse events were documented on study records as they were observed.

The unit of observation and statistical analysis was the individual dog. All animals were included in the analyses. Data were compared using descriptive statistics, tables, and figures as appropriate.

### Results and Discussion

During acclimation (day −6), the body weight of dogs ranged from 10 kg to 13.8 kg. Ten instances of vomiting in eight dogs were documented during the seven days of capsule dosing, most prevalently in groups T3, T5 and T5c (Table 12). The prevalence of vomiting was significantly reduced with use of the liquid formulation. This suggested that the major cause of vomiting was the dosing of multiple capsules. However, given that vomiting was also noted in group T0, this suggests that vomiting was not associated with test article administration. All dogs accepted the liquid formulation of test article willingly.

TABLE 12
Summary of abnormal clinical observations.

| | | Number of | | |
|---|---|---|---|---|
| System | Observation | instances | Group | Study day |
| Eye | Blepharospasm | 1 | T5 | 25 |
| | Conjunctival hyperemia | 5 | T1 | 17 to 21 |
| | | 19 | NA | −7 to 13 |
| | | 2 | T5 | 25 to 26 |
| | Epiphora | 1 | T0 | 22 |
| | | 2 | T1 | 23 to 24 |
| | | 17 | NA | −1 to 28 |
| | | 1 | T3 | 0 |
| | | 6 | NA | −7 to 23 |
| | Muco-purulent | 2 | T1 | 17 to 28 |
| | discharge | 1 | T5 | 25 |
| | Swelling | 1 | T5 | 25 |
| Feces | Mucus in feces | 1 | T3 | 22 |
| | Fecal score: 7 | 1 | T0 | 1 |
| Vomit | Capsule/TA powder in | 1 | T5 | 1 |
| | vomit | 1 | T5c | 1 |
| | | 2 | T5c | 2, 3 |
| | | 2 | T3 | 3, 6 |
| | | 1 | T5 | 6 |
| | | 1 | T3 | 6 |
| | Partially digested | 2 | T3 | 0, 4 |
| | food | 1 | T3 | 19 |
| | Bile | 2 | T5 | 12, 18 |
| | | 1 | T1 | 14 |
| | | 1 | T3 | 19 |
| | | 1 | T0 | 24 |
| | | 1 | T3 | 26 |

NA, not available

Abnormal observations were only reported in eyes and included blepharospasm (1 instance, 1 dog affected), conjunctival hyperemia (26 instances, 3 dogs affected), epiphora (28 instances, 5 dogs affected), muco-purulent discharge (3 instances, 2 dogs affected), swelling (1 instance, 1 dog affected). One instance each of mucus in feces (1 dog affected) and fecal score of 7 (1 dog affected) was reported (Table 12).

Some of these observations were recurrent throughout the study, including acclimation. All documented abnormal observations were mild, transient, and did not require veterinary intervention. There were no serious adverse events. There was no dose group effect on any abnormalities noted during these observations.

Mild azotemia proteinuria was reported in 1 dog each in groups T1 and T5c on day 28. Dogs had a mild azotemia (increased urea, creatinine), and mild proteinuria, with normal urine specific gravity. There were no noteworthy hematological findings.

There was no dose group effect on any abnormalities noted during these observations. Findings were either incidental, of known etiology, reflective of pre-existing processes or attributable to clinical procedures unrelated to dosing.

Overall, there were no results suggestive of a test article effect of clinical or toxicological significance. No clinically significant abnormalities were noted which warranted exclusion from the study. Subsequent physical examinations at days 14, 15, and 28 revealed typical dermatological, otic, and dental findings, none of which was drastically different from the physical examination performed during acclimation.

There was no effect of dose group on body weight; therefore, there was no clinically or toxicologically relevant effect of test article on this variable. There was no effect of dose group on food consumption. Feed consumption in all dose groups was consistent with that of normal, healthy dogs.

Results showed no clinically significant abnormalities after oral administration of test article in dogs in this study. Few instances of vomiting were reported in dogs which might be potentially due to capsule formulation as prevalence of vomiting was resolved with change in formulation. Abnormal clinical observation was reported in eyes and included Epiphora, Conjunctival Hyperemia and Muco-purulent discharge. These ocular morbidities are commonly reported in dogs (17-19). These ocular observations were mild, transient, and did not require veterinary intervention. Moreover, these observations were not dose dependent which further proves that they were unlikely to be associated with administration of test article.

Mild azotemia proteinuria was reported in 2 dogs. Both instances can be deemed spurious. Renal involvement is highly unlikely in absence of other clinical or urinary signs. Also, both cases of proteinuria can be false, and caused by a minor trauma during catheterization. As with other cases this observation was also not dose dependent and therefore, it is not likely to be associated with the test article.

There was no abnormality reported in observations pertaining to haematology, physical, body weight or food intake. These results are consistent with safety outcome of other probiotics in dogs (13,20). With a well-tolerated safety profile, Enterococcus faecium postbiotic metabolites with their natural, highly targeted ability and their combination with probiotics providing potentially added benefit can be robust products for companion animals.

### REFERENCES

1. Weese J S, and Anderson M E. Preliminary evaluation of Lactobacillus rhamnosus strain GG, a potential probiotic in dogs. The Canadian Veterinary Journal 2002; 43:771.
2. Baillon M L A, Marshall-Jones Z V, and Butterwick R F. Effects of probiotic Lactobacillus acidophilus strain DSM13241 in healthy adult dogs. American journal of veterinary research 2004; 65:338-343.
3. Rastall R A. Bacteria in the gut: friends and foes and how to alter the balance. The Journal of nutrition 2004; 134:2022S-2026S.
4. Bayoumi, M A, Griffiths M W. In vitro inhibition of expression of virulence genes responsible for colonization and systemic spread of enteric pathogens using Bifidobacterium bifidum secreted molecules. International journal of food microbiology 2012; 156:255-263.
5. Mundi A, Delcenserie V, Amiri-Jami M, Moorhead S, Griffiths M W. Cell-free preparations of Lactobacillus acidophilus strain La-5 and Bifidobacterium longum strain NCC2705 affect virulence gene expression in Campylobacter jejuni. Journal of food protection 2013; 76:1740-1746.
6. Medellin-Pena M J, Griffiths M W. Effect of molecules secreted by Lactobacillus acidophilus strain La-5 on Escherichia coli O157: H7 colonization. Appl. Environ. Microbiol. 2009; 75:1165-72.
7. Aguilar-Toala J E, Garcia-Varela R, Garcia H S, Mata-Haro V, Gonzalez-Cordova A F, Vallejo-Cordoba B, Hernandez-Mendoza A. Postbiotics: An evolving term within the functional foods field. Trends in Food Science & Technology. 2018.
8. Nordeste R, Tessema A, Sharma S, Kovač Z, Wang C, Morales R, Griffiths M W. Molecules produced by probiotics prevent enteric colibacillosis in pigs. BMC veterinary research. 2017; 13:335.
9. Loh T C, Choe D W, Foo H L, Sazili A Q, Bejo M H. Effects of feeding different postbiotic metabolite combinations produced by Lactobacillus plantarum strains on egg quality and production performance, faecal parameters and plasma cholesterol in laying hens. BMC veterinary research. 2014; 10:149.
10. Sanders M E, Klaenhammer T R. Invited review: the scientific basis of Lactobacillus acidophilus NCFM functionality as a probiotic. Journal of dairy science. 2001; 84:319-331.
11. Altermann E, Russell W M, Azcarate-Peril M A, Barrangou R, Buck B L, McAuliffe O, Souther N, Dobson A, Duong T, Callanan M, Lick S. Complete genome sequence of the probiotic lactic acid bacterium Lactobacillus acidophilus NCFM. Proceedings of the National Academy of Sciences. 2005; 102:3906-3912.
12. Doron S, Snydman D R. Risk and safety of probiotics. Clinical Infectious Diseases. 2015; 60:S129-S134.
13. Kelley R L, Park J S, O'Mahony L, Minikhiem D, Fix A. Safety and tolerance of dietary supplementation with a canine-derived probiotic (Bifidobacterium animalis strain AHC7) fed to growing dogs. Vet Ther. 2010; 11:E1-4.
14. Kayser F H. Safety aspects of enterococci from the medical point of view. International journal of food microbiology. 2003; 88:255-262.
15. Gao J, Li Y, Wan Y, Hu T, Liu L, Yang S, Gong Z, Zeng Q, Wei Y, Yang W, Zeng Z. A novel postbiotic from Lactobacillus rhamnosus GG with a beneficial effect on intestinal barrier function. Front Microbiol. 2019; 10.
16. Kareem K Y, Loh T C, Foo H L, Akit H, Samsudin A A. Effects of dietary postbiotic and inulin on growth performance, IGF1 and GHR mRNA expression, faecal microbiota and volatile fatty acids in broilers. BMC Vet Res. 2016; 12:163.
17. Krastev S. Retrospective study on the prevalence of uveitis in dogs. Animal studies & Veterinary medicine 2015; Volume V.
18. Chen J, Dinh T, Woodward D F, Holland J M, Yuan Y D, Lin T H, Wheeler L A. Bimatoprost: mechanism of ocular surface hyperemia associated with topical therapy. Cardiovascular drug reviews. 2005; 23:231-46.
19. Komnenou A T, Thomas A L, Kyriazis A P, Poutahidis T, Papazoglou L G. Ocular manifestations of canine transmissible venereal tumour: a retrospective study of 25 cases in Greece. Veterinary Record. 2015; 176:523.
20. European Food Safety Authority (EFSA). Opinion of the Scientific Panel on additives and products or substances used in animal feed (FEEDAP) on the safety of product “MLB” Lactobacillus acidophilus for dogs. EFSA Journal. 2004 April; 2(4):52.

### Example 3: Ygia¹⁴ Improves Stool Quality and can Resolve Diarrhea in Dogs

### Introduction

Diarrhea is a common gastrointestinal ailment reported in companion animals, which can be defined as an increase in fecal water content; usually leading to changes in fecal volume, fluidity, and frequency of defecation (Hall, 2009). There is an unmet need for an effective alternate treatment option which should not only prevent diarrhea but should also improve gastrointestinal dysbiosis. Due to a unique mode of action, probiotic supplementation can be useful to resolve gastrointestinal problems. However, the mode of action of a probiotic is strain dependent. Ygia¹⁴ is a combination of live Enterococcus faecium (200 million CFU/dose) and postbiotic metabolites produced during a proprietary fermentation process of the same strain. Due to the in vitro and in vivo efficacy of postbiotic metabolites and the ability of probiotics to rebalance the gastrointestinal microflora, Ygia¹⁴ could be used as an effective non-antibiotic anti-infective to manage diarrhea symptoms in dogs. Here, we report results from Ygia¹⁴ administration in dogs with acute or chronic diarrhea.

### Case Description

Veterinarians in Southern Ontario were contacted by MicroSintesis to dispense Ygia¹⁴ and 26 veterinary practices participated. The veterinarians were given Ygia¹⁴ to try on individual cases at their own discretion and no attempt was made to restrict its combination with other products. The veterinarians contacted the owner with a follow-up call and asked questions related to fecal score, number of treatments before fecal quality improvement and number of treatments until stool returned to normal. The fecal quality was evaluated using 7-point scale fecal scoring ranging from very hard and dry (score of 1) to watery with no texture (score of 7). The improvement in fecal score was assessed based on the fecal score before Ygia¹⁴ administration and diarrhea was considered resolved if the fecal score reached the normal fecal score for the individual dog. Data for 92 dogs were analyzed and included 58 dogs with acute (abrupt onset of 3 or more loose stools per day) and 34 with chronic diarrhea (diarrhea lasting for at least 4 weeks) as indicated by the veterinarians' records. Dogs ranged in age from 2 months to 16 years with a median age of 5 years and an average of 5.6 years. Their weight ranged from 2 to 88 kg with a median weight of 12 kg and an average weight of 19 kg. Ygia¹⁴ was administered based on weight; with 38 dogs receiving the lower dose (1 cc), 26 the large dose (2.5 cc) and 28 administered 2 of the 2.5 cc doses daily for 14 days.

The most common dog breeds were terriers, mixed breeds and working dogs representing 54% of the dogs. In total, 30 different breeds were represented. In 8 of the records, blood was found in the feces before the initial visit, while the other 84 exhibited varying severity of diarrhea from mild to severe. During the follow-up discussion, the pet owners indicated the number of Ygia¹⁴ doses needed to see an improvement in fecal score. Of the 54 owners that responded, 81.1% indicated that the fecal score improved within 72 hours. The owners also indicated the numbers of days until the diarrhea symptoms were completely resolved and 75.4% indicated the fecal score was back to normal within 72 hours.

The previous medication for diarrhea or gastrointestinal upset was recorded, 53 dogs did not previously receive any medication while 39 had received an antibiotic, a probiotic or a combination of the two, without resolution of diarrhea. The data in Table 13 indicates that Ygia¹⁴ was most often given to dogs with acute diarrhea that had not been given antibiotics previously. Fifty of the 92 dogs treated were in this category and 34 of those 50 dogs (68%) had their symptoms of diarrhea improved or resolved. The next highest category was dogs that were only given Ygia¹⁴. There were 35 dogs that were in this category of which 23 had their diarrhea symptoms improved or resolved. In both groups, Ygia¹⁴ improved or resolved diarrhea incidence in 68% and 66% of the cases, respectively. The cause of diarrhea is unknown in these cases and they may not be the result of a bacterial infection, which is the primary target for Ygia¹⁴. The next largest category consisted of dogs in which Ygia¹⁴ was administered concurrently with an antibiotic. The most common antibiotics that were used were metronidazole, which is also used to treat Giardia, a common parasite, and tylosin, which is a broad-spectrum antibiotic. In 19 of the 23 cases, Ygia¹⁴ improved or resolved the diarrhea symptoms, however, within this group it is unclear if the diarrhea symptoms were improved or resolved by the antibiotic or Ygia¹⁴. In the last category, which was arguably the most interesting, Ygia¹⁴ was used when a previous administration of antibiotic was not effective. Although this was a small group, 6 of the 8 dogs had their symptoms either improved or resolved by Ygia¹⁴. These cases indicate that there are uses for Ygia¹⁴ when antibiotics are not effective, possibly the result of the diarrhea being caused by antibiotic resistant bacteria.

TABLE 13
Efficacy of Ygia¹⁴ in the treatment of acute diarrhea in
dogs distributed between with and without antibiotic use.

| | Pre- | Pre- | Con- | Con- | |
|---|---|---|---|---|---|
| | vious | vious | current | current | All |
| | Anti- | Anti- | Anti- | Anti- | Acute |
| | biotic | biotic | biotic | biotic | Cases |
| Treatment | Yes | No | Yes | No | — |
| Number of Improve | 6 | 34 | 19 | 23 | 42 |
| or Resolved | | | | | |
| Total Cases | 8 | 50 | 23 | 35 | 58 |
| Percentage | 75 | 68 | 83 | 66 | 72 |
| Improved/Resolved | | | | | |

The data in Table 14 indicates that for chronic diarrhea cases, Ygia¹⁴ was most often used when an antibiotic was previously used. In 20 of the 22 (91%) chronic cases, Ygia¹⁴ was able to improve or resolve the diarrhea symptoms when a previous antibiotic was not effective. Whereas, out of 12 cases where antibiotic was not used previously, Ygia¹⁴ was able to improve or resolve the diarrhea symptoms in 9 (75%) cases. In the cases where Ygia¹⁴ was not used concurrently with an antibiotic, Ygia¹⁴ was able to improve or resolve the diarrhea symptoms in 16 of the 21 (75%) dogs. When Ygia¹⁴ was used concurrently with antibiotics, it was able to improve or resolve chronic diarrhea symptoms in 12 of the 13 (92%) cases.

TABLE 14
Efficacy of Ygia¹⁴ in the treatment of chronic diarrhea in
dogs distributed between with and without antibiotic use.

| | Pre- | Pre- | Con- | Con- | |
|---|---|---|---|---|---|
| | vious | vious | current | current | All |
| | Anti- | Anti- | Anti- | Anti- | Acute |
| | biotic | biotic | biotic | biotic | Cases |
| Treatment | Yes | No | Yes | No | — |
| Number of Improve | 20 | 9 | 12 | 16 | 28 |
| or Resolved | | | | | |
| Total Cases | 22 | 12 | 13 | 21 | 34 |
| Percentage | 91 | 75 | 92 | 76 | 82 |
| Improved/Resolved | | | | | |

In general, Ygia¹⁴ was effective in treating both chronic (82%) and acute (72%) diarrhea. Additionally, in some cases, Ygia¹⁴ was able to improve or resolve the diarrhea symptoms when previous antibiotic therapies were not effective. Additionally, Ygia¹⁴ was mostly well-tolerated and had no safety concerns except in 1 case when a Chihuahua experienced a painful and distended abdomen, most likely due to potential whey sensitivity.

### Discussion

Postbiotic metabolites produced by Enterococcus faecium can reduce the virulence of pathogenic bacteria. Virulence factors are involved in bacterial attachment, invasion, and toxin production and are a part of the bacterial infection mechanism. In the present case studies, dogs that had either chronic or acute diarrhea were given Ygia¹⁴ and the data indicated that Ygia¹⁴ could improve or resolve the diarrhea symptoms in 72.4% of acute cases and 82.4% of chronic cases. The cause of the diarrhea was unknown in each case and could be caused by stress, viral, bacterial or parasitic infections, for example. Since postbiotic metabolites were developed to be effective against bacteria, the low efficacy that was observed in certain situations may be the result of diarrhea not being caused by a bacterial infection.

The data presented in Tables 13 and 14 suggest that Ygia¹⁴ is effective in both chronic and acute diarrhea, however, results appeared to be better in chronic cases compared to acute diarrhea, which may be a result of acute diarrhea being caused by non-bacterial agents such as parasites or some external stress factor. Moreover, Ygia¹⁴ appeared to be more effective in chronic cases when an antibiotic had previously been used but could not completely treat diarrhea, in these cases Ygia¹⁴ was effective in 20 of 22 cases. Although the underlying causes of the diarrhea symptoms in these cases are unknown, they are typically the result of a change in the gastrointestinal microbiome (Suchodolski et al., 2012). Nevertheless, Ygia¹⁴ was able to alleviate the symptoms even when an antibiotic was not completely effective. It is difficult to speculate on the actual cause of better efficacy of Ygia¹⁴ over antibiotic, but it may be due to the presence of an antibiotic resistant aetiological agent or the improper selection of the antibiotic.

Veterinarians use antibiotics because they believe they will work quickly and effectively and, as a result, they expect the same from antibiotic alternatives. The data from these case studies indicated that in 81.1% of dogs Ygia¹⁴ improved the fecal score within 72 hours and in 75.4% of the cases the diarrhea was completely resolved. There were also a few cases where the symptoms were improved after one dose; however, those were likely exceptional cases. These case studies support the use of Ygia¹⁴ as an effective alternative to antibiotic treatment, especially when antibiotic resistance may be a concern. The use of efficacious antibiotic alternatives such as postbiotic metabolites helps reduce the overall use of antibiotics and minimizes the global impact of antibiotic resistance on animal and human health.

### REFERENCES

Hall E D (2009) Canine diarrhea: a rational approach to diagnostic and therapeutic dilemmas. In. pract. 31 (1):8-16.
Bayoumi M A, & Griffiths M W (2010) Probiotics down-regulate genes in Salmonella enterica Serovar Typhimurium pathogenicity islands 1 and 2. J. Food Prot. 73 (3):452-460.
Medellin-Pena M J, Wang H, Johnson R, Anand S, and Griffiths M W (2007) Probiotics affect virulence-related gene expression in Escherichia coli O157:H7. Appl. Environ. Microbiol. 73 (13):4259-4267.
Kim Y, Lee J W, Kang S G, Oh S, and Griffiths M W (2012) Bifidobacterium spp. influences the production of autoinducer-2 and biofilm formation by Escherichia coli O157:H7. Anaerobe. 18 (5):539-545.
Medellin-Pena M J, and Griffiths M W (2009) Effect of molecules secreted by Lactobacillus acidophilus strain LA5 on Escherichia coli O157:H7. Appl. Environ. Microbiol. 75 (4):1165-1172
Yun B, Oh S, and Griffiths M W (2014) Lactobacillus acidophilus modulates the virulence of Clostridium difficile. J Dairy Sci. 97 (8):4745-4758.
Mundi A., Delcenserie V., Amiri-Jami M., Moorhead S., and Griffiths M. W (2013) Cell-free preparations of Lactobacillus acidophilus strain La-5 and Bifidobacterium longum strain NCC2705 affect virulence gene expression in Campylobacterjejuni”, J Food Prot. 76 (10):7401746.
Suchodolski J S, Markel M E, Garcia-Mazcorro J F, Unterer S, Heilmann R M E Dowd, P. Kachoo, I. Ivanov, Y. Minamoto, E. M. Dillman, J. M. Steiner, A. K. Cook, and L. Toresson S (2012) The fecal microbiome in dogs with acute diarrhea and idiopathic inflammatory bowel disease. PLoS One 7 (12):e51907.

### Example 4: Probiotic Disruption of Quorum Sensing Reduces Carotenoid Production and Increases Cefoxitin Sensitivity in Methicillin Resistant Staphylococcus aureus

### Abstract

Antimicrobial resistance is a growing threat to food safety, medical advancement and overall global health. Methicillin resistant Staphylococcus aureus (MRSA) is typically a commensal species that, given an opportunity to establish an infection, transforms into a formidable pathogen with high rates of mortality and morbidity. Therefore, it is globally recognized that new therapies to combat this particular pathogen are desperately needed. A potential strategy in combating MRSA resistance and infections is the development of alternative therapeutics that interfere with bacterial quorum sensing (QS) systems involved in cell-to-cell communication. QS systems are crucial in the regulation of many virulence traits in MRSA, such as methicillin resistance, exotoxin and surface protein expression, antioxidant production and immune cell evasion. Based on our previous research, in which we have shown that probiotic bioactive metabolites act as novel QS-disrupting compounds, we propose herein that the same probiotic compounds can be used as adjuvants in tandem with antibiotics, such as a β-lactam antibiotic, cefoxitin, to “re-sensitize” MRSA clinical isolates to cefoxitin. Moreover, we show that these probiotic compounds are able to reduce production of carotenoids in active cultures of MRSA, resulting in decreased resistance to hydrogen peroxide cytotoxicity.

### Introduction

One such novel alternative that has recently emerged as a contender to combat pathogenic bacteria is the use of probiotic bacteria and their metabolites that act as QS disruptors⁸. Although scientific evidence to support claims of the benefits of probiotics on human and animal health is scarce, recent work has elucidated several mechanisms by which probiotics act to directly interfere with pathogen virulence. Metabolic peptides produced by Lactobacillus acidophilus have been shown to reduce virulence in pathogenic bacterial strains by reducing their use of QS to sense and communicate in their environment: an important contributor to the pathogen's ability to produce toxins, invade host cells, evade immune cells, and form biofilms^{11,12}. Metabolites isolated from the cell free spent medium (CFSM) of Bifidobacterium cultures have also been shown to down regulate the main regulatory genes controlling the virulence factors necessary for attachment and adhesion in Salmonella enterica serovar Typhimurium and Enterohaemorrhagic Escherichia coli O157:H7^{13,14}. Notably, it has been shown in vivo that the probiotic Bacillus subtilis produces quorum quenching metabolites that significantly contributed to the exclusion of the commensal pathogen S. aureus in the gut within rural Thai populations¹⁵.

Staphylococcus aureus is a pathogen of serious concern. Gram-positive bacteria utilize autoinducing oligopeptides in intercellular QS-controlled gene expression systems, and communication via these impermeable autoinducers is mediated by specialized transporters. The accessory gene regulator (agr) QS system is one of the most well-described communication systems comprising a two-component system in S. aureus¹⁶. Agr is greatly influenced by cell population density and regulates virulence expression as required by S. aureus in the various stages of infection¹⁷. It has also been shown that the response regulator of the agr system (agrA) possesses an oxidation-sensing ability that is critical in S. aureus defense against oxidative stress and immune cell evasion¹⁸, and that the agr system is essential in density-dependent regulation of exotoxin and cell surface protein expression^{19,20}. Previous research has also shown that the agr system displays a significant regulatory effect on the mecA gene that controls methicillin resistance in highly virulent community-acquired methicillin resistant S. aureus (MRSA)^{21,22,23}. Thus, it has been proposed that the agr system should be considered as a significant candidate for QS disruption and novel therapeutic intervention in S. aureus⁹; furthermore, an intriguing idea is the combination of QS disruptors as non-antibiotic adjuvants in a combination therapy with established antibiotics.

### Methods

Probiotic bacterial strain. A frozen glycerol stock culture of Enterococcus faecium was obtained from −80° C. storage from the Canadian Research Institute for Food Safety (University of Guelph, Ontario, Canada); this stock culture was originally prepared from a late log-phase growth from a Difco™ MRS broth culture (Becton, Dickinson and Company, Sparks, Md., USA). This stock probiotic strain was used to produce all bioactive materials containing probiotic metabolites used in this study.

Staphylococcal bacterial strains. Two clinical isolates of S. aureus bacteria were obtained from the Atlantic Veterinary College (AVC) at the University of Prince Edward Island (Charlottetown, PE, Canada). The presence of methicillin resistance via the SCC mecA gene pathway in both strains was confirmed with an oxacillin disk diffusion method performed by AVC staff, as well as by individual minimum inhibitory testing with the β-lactam antibiotic cefoxitin where S. aureus strains were deemed methicillin resistance-positive if the MIC ≥8 μg/mL. The presence of the mecA gene was finally confirmed by positive qPCR testing with the following primer pair (5′ to 3′): forward primer AACAGGTGAATTATTAGCACTTGTAAG and reverse primer ATTGCTGTTAATATTTTTTGAGTTGAA²⁹. The first strain alias was specified as MRSA Livestock Associated (LA) 414M SPA t034 (“MRSA LA”), and the second strain alias was specified as MRSA 81 M SPA t008 (“MRSA 81 M”). The clinical isolates were maintained on sheep blood agar slants, and a glycerol stock of each strain was made from a late log-phase culture grown from multiple colonies and stored at −80° C. A loop culture from the glycerol stocks of each respective MRSA strain was then used to inoculate an overnight culture in in BBL™ cation-adjusted Mueller Hinton media (Becton, Dickinson and Company, Sparks, Md., USA), from which LB streak plates were made following 18-20 h incubation; these streak plates were used to inoculate starting cultures for all assays. Cultures of MRSA were grown in cation-adjusted Mueller Hinton media for all experimental assays.

Preparation of probiotic bioactive material. The contents of the Enterococcus faecium stock vial were defrosted, and a loop culture was inoculated in 200 mL of 0.22 μM filter-sterilized modified MRS medium (VWR International, Mississauga, Ontario, Canada). This bottle culture was sealed and incubated without shaking at 37° C.±1° C. for 18 h. Following incubation, this culture was used to inoculate 4 L of chemically defined medium (CDM) with 200 g Whey Protein Isolate and 25 g lactose. The vessel culture was then incubated statically at 37° C.±1° C. for 48 h. Following incubation, the cells were isolated from the liquid phase by centrifugation at 12,000×g for 30 min at 4° C. (Avanti J-20 XPI, Beckman Coulter, Canada). The cell-free supernatant containing the probiotic metabolites was then frozen at −80° C. and freeze dried. The dried cell-free supernatant was kept in long-term storage in powder form at −20° C. until needed.

Resuspension of probiotic bioactive material. The resuspension of the dried cell-free supernatant was only performed prior to each experiment to maintain optimal activity of the metabolites. The method is as follows. The dried supernatant was weighed out and resuspended in cation-adjusted Mueller Hinton media at the desired concentration (5, 30 and 60 mg/mL). The pH of the resuspended material was checked with an Accumet® AE150 pH meter (Fisher Scientific) and adjusted as needed with 1.0 N sodium hydroxide solution to maintain stable test conditions at a pH of 7.3±0.1. The resuspended solution was then centrifuged at 5,000×g for 15 min at room temperature with a Centrifuge 5804 (Eppendorf). The remaining liquid was separated from the debris pellet and filtered through a Supor®-800 0.45 μM membrane filter (Pall Corporation) with a 40/35 Synthware vacuum filtration apparatus (Kemtech America) to remove remaining colloidal material. The filtrate was then filter-sterilized using a Basix™ 25 mm 0.2 μM syringe filter (Thermo Fisher Scientific). The samples were aliquoted into smaller 5-10 mL volumes to minimize freeze-thaw manipulations of the liquid bioactive samples as they were stored at −20° C. in sterile conical tubes until needed for the experiments.

Preparation of Molecular Weight Cut Off (MWCO 3000) fractions. To better understand the metabolite bioactives' sizes, an Amicon® Ultra 15 mL centrifugal filter with a Molecular Weight Cut Off (MWCO) of 3000 Da (Millipore Sigma) was used for ultrafiltration of the resuspended probiotic cell-free supernatant. Following 0.2 μM filtration to remove remaining debris, 10 mL of the resuspended supernatant was added to the MWCO 3000 centrifugal filter tube and centrifuged at 5,000 rpm for 1 hour. The MWCO 3000 filtrate was collected; the remaining retentate fraction was collected by rinsing the filter head twice with 10 mL of cation-adjusted Mueller Hinton media for each rinse. Following the two rinses, an additional 10 mL of media was used to resuspend and collect the retentate. All collected fractions were then filter-sterilized with a 0.2 μM syringe filter to remove any contamination. The MWCO 3000 liquid retentate and filtrate solutions were stored in sterile centrifuge tubes at −20° C.

Preparation of cefoxitin for MIC testing. The antibiotic selected for minimum inhibitory concentration (MIC) testing in the two MRSA strains was cefoxitin as outlined in the Clinical and Laboratory Standards Institute (CLSI) guidelines for MIC testing of Staphylococcal species³⁰. Cefoxitin has been shown to strongly upregulate the SSC mecA pathway for antibiotic resistance in MRSA strains³¹ and was therefore a superb antibiotic candidate for this study. Cefoxitin in powder form (Alfa Aesar) was weighed with an analytical balance (Mettler Toledo) and resuspended in methanol at 10 mg/mL and stored at −20° C. until used in the MIC and FIC test assays.

Minimum Inhibitory Concentration (MIC) testing. MIC testing was performed with respect to the Clinical and Laboratory Standards Institute (CLSI) guidelines for MIC testing of Staphylococcal species³⁰. Overnight cultures of each respective clinical MRSA strain were diluted 1000-fold to obtain approximately 10⁶ CFU/mL as the starting inoculate. Dilution ranges for cefoxitin were from 5 μg/mL to 100 μg/mL. Clinical MRSA strain MICs with cefoxitin were determined by broth microdilution with cefoxitin in cation-adjusted Mueller Hinton media in Costar® clear 96-well standard flat-bottom microplates (Corning®). Media-only aliquots were added as sterility checks and were also used as background controls; background controls were subtracted from the turbidity measurements to account for media background. All test sample volumes were 200 μL per well with technical duplicates. Following sample preparation and aliquoting, the MIC test plates were sealed with parafilm and incubated at 35° C.±1° C. and 200 rpm shaking for 24 h in a VWR® Incubating Mini Shaker and temperature was monitored with a Fisherbrand™ digital thermometer (Fisher Scientific). Following incubation, the plates were cooled to room temperature and turbidity was measured using a SpectraMax M2 microplate reader (Molecular Devices) at a wavelength of 600 nm. The lowest concentrations of cefoxitin that resulted in either 90% or greater reduction in turbidity compared to the respective positive-growth controls were defined as the MIC. All MIC tests were performed in three biological replicates.

Fractional Inhibitory Concentration (FIC) testing. The FIC is a mathematical expression used to describe the effects of combinations of two or more antibiotics or of an antibiotic and a non-antibiotic compound; effects may be described as antagonistic, indifferent, additive or synergistic as defined by the FIC index³². Checkerboard MIC analyses were performed for three pre-determined probiotic bioactive material concentrations to determine the synergistic, additive, or antagonistic combinatory effects with cefoxitin against the clinical MRSA strains. Dilution ranges of the bioactive material were 5, 30 and 60 mg/mL of freeze dried cell-free supernatant. Dilution ranges for cefoxitin were from 5 μg/mL to 100 μg/mL. The FIC tests were performed identically to the MIC test method outlined above.

FIC index determination. The FIC value was determined for the combination of each respective concentration of bioactive material in conjunction with the dilution range of cefoxitin for each respective clinical MRSA strain. The FIC index was observed as described by EUCAST³². Equation 1 and 2 was used to determine the FIC values for cefoxitin and the bioactive material, and Equation 3 was used to calculate the FIC index to determine the combinatory effect of the two components:

$${FIC}_{A} = \frac{{MIC}_{C}}{{MIC}_{A}} \qquad Equation 1$$

$${FIC}_{B} = \frac{{MIC}_{C}}{{MIC}_{B}} \qquad Equation 2$$

$${FIC}_{i} = \sum FIC = {FIC}_{A} + {FIC}_{B} \qquad Equation 3$$

Where FIC_{A} and FIC_{B} are the FIC values for drug A and drug B, respectively. MIC_{A} and MIC_{B} are the respective MICs of drug A and drug B alone. MIC_{C} is the MIC of drug A and drug B in combination. The FIC index (FICᵢ) is the sum of FIC_{A} and FIC_{B}. The criteria index for determining the results of the FICᵢ calculations between drugs A and B is as follows: a synergistic outcome is defined as the combination of two antibiotics or an antibiotic and a non-antibiotic compound that exceed the observed additive effects of the individual components 0.5), an additive outcome is the sum of the effects of the individual components (>0.5-1.0), an indifferent outcome is equal to the effect observed from the most active component (>1.0-2.0), and an antagonistic outcome is observed when the combination of two compounds have a reduced effect in comparison to the most active individual component (>2.0)³².

MWCO 3000 fraction MIC testing. Following MIC checkerboard testing, as a clinical strain with a high MIC, MRSA 81 M was selected for testing of ultrafiltration fractions of the bioactive material to better elucidate the bioactives' sizes. Only MRSA 81 M was selected for testing as it appeared to have greater sensitivity to the bioactive material, and showed synergistic FIC values at all tested concentrations whereas MRSA LA did not express a similar pattern; the concentration of 30 mg/mL bioactive material was selected as it was the lowest concentration that had an FIC value well under 0.5. The MWCO 3000 MIC tests were performed identically to the MIC test method outlined above.

Carotenoid measurements. Staphylococcal carotenoid concentrations were extracted using a previously established methanol extraction protocol²⁴. Each respective MRSA strain was inoculated into 10 mL of cation-adjusted Mueller Hinton media and 10 mL of cation-adjusted Mueller Hinton media supplemented with 30 mg/mL of probiotic bioactive material. The samples were grown at 37° C.±1° C. and 200 rpm shaking for 24 h. Following incubation, the samples were centrifuged at 4,000 rpm for 15 mins, washed in 1× Dulbecco's PBS solution (VWR Life Science), transferred to clean 1.5 mL centrifuge tubes and then resuspended in 500 μL of methanol. The samples were incubated in a warming block at 40° C. for 45 minutes. The samples were then centrifuged at 10,000 rpm for 5 mins to pellet the remaining cell debris. The supernatant was pipetted in 200 μL (with duplicates) into a standard 96-well plate and the absorbance at a wavelength of 450 nm (A₄₅₀) was measured with a SpectraMax M2 microplate reader (Molecular Devices).

Oxidant susceptibility assays. The ability of the clinical MRSA strains to withstand oxidant killing was analyzed using a previously established protocol²⁴. Oxidant susceptibility assays were performed in 1×PBS solution. Each respective MRSA strain was inoculated into 10 mL of cation-adjusted Mueller Hinton media and 10 mL of cation-adjusted Mueller Hinton media supplemented with 30 mg/mL of probiotic bioactive material. The samples were grown at 37° C.±1° C. and 200 rpm shaking for 24 h. Following incubation, the samples were centrifuged at 4,000 rpm for 15 mins, washed in 1× Dulbecco's PBS solution (VWR Life Science), and resuspended at about 10⁹-10¹⁰ CFU/mL in 6 mL of 1×PBS supplemented with 1.5% v/v hydrogen peroxide. The starting inocula concentrations were plated on standard LB agar plates. The cultures were then incubated at 37° C.±1° C. without shaking for 1 h. Dilutions of the respective samples were performed, plated on standard LB agar plates, and incubated for 15-20 h at 37° C. to enumerate the surviving CFU/mL of the MRSA cultures.

Statistical methods. All statistical analyses were performed as follows. MIC data are presented as the mean in terms of percent growth inhibition. Cytotoxicity and absorbance data are presented as the median with whisker box plots depicting the minimum and maximum. All replicates (n) are biological (with technical duplicates for each). Wilcoxon Signed Rank for paired statistical testing and Mann Whitney U between two samples was performed using statistical hypothesis testing.

### Results and Discussion

It is described herein that QS disruptors produced by probiotic bacteria could be utilized in combating antimicrobial resistance by aiding ineffectual antibiotics to return to more efficacious states against certain MRSA strains. Additionally, it is proposed that, due to the overarching regulatory presence of the agr system in MRSA, the disruption of QS-regulated factors that greatly contribute to MRSA virulence (e.g. oxidant survival) may also be achieved by these probiotic metabolites in parallel. To analyze the potential capabilities of certain probiotic compounds in virulence reduction, the CFSM of the probiotic bacteria Enterococcus faecium was selected. Moreover, two clinical isolate strains of MRSA were selected for comparison of reducing several key virulence factors well described in MRSA populations²⁰: staphyloxanthin and carotenoid synthesis²⁴, increased hydrogen peroxide survival and resistance to oxidant killing^{24,25}, possession of the mobile genetic element Staphylococcal cassette chromosome (SCC) containing the genes for antibiotic resistance machinery and regulation and expression of the mecA gene located on the SCC^{9,17, 21,22,23}.

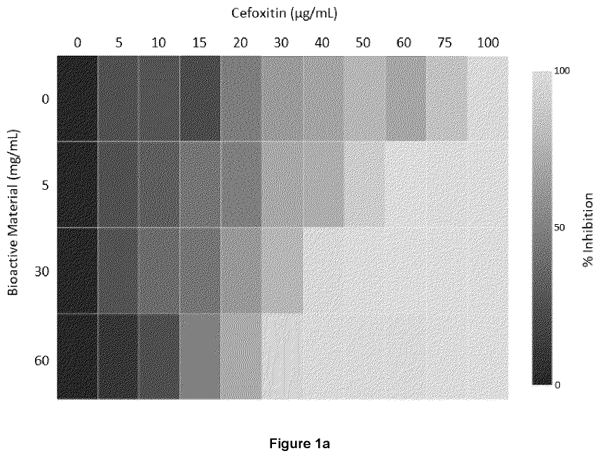

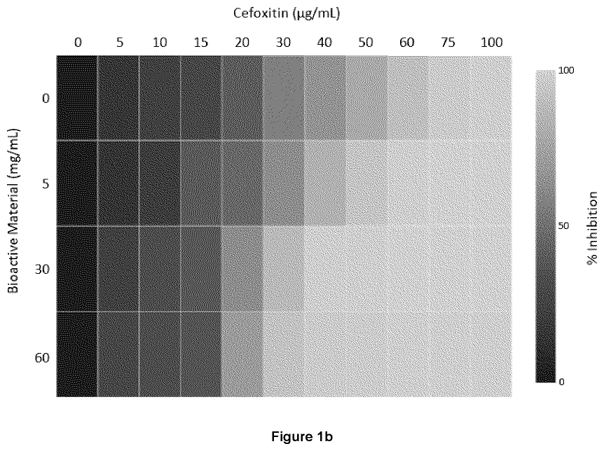

To probe this probiotic space, the individual minimum inhibitory concentrations (MIC) of two clinical MRSA strains, strain 81M (“MRSA 81M”) and strain LA414M (“MRSA LA”) were first determined against the β-lactam antibiotic cefoxitin, a strong inducer of β-lactamase enzymes (with MICs of 100 μg/mL and 60 μg/mL, for each strain, respectively). The addition of three concentrations of filter-sterilized cell-free supernatant metabolites were added in checkerboard fashion to a range of cefoxitin concentrations (0-100 μg/mL) with equal starting inocula of each respective MRSA strain. The results indicate a concentration-dependent decrease in the minimum concentration of cefoxitin required to reduce the MRSA cell density by more than 90% when incubated with bioactive metabolites for 24 h at 35° C.±1° C. (FIG. 1a,b). The Fractional Inhibitory Concentration (FIC) index was used to evaluate the potency of combining a non-antimicrobial compound with cefoxitin²⁶. The criteria index for determining the results of the FIC calculations between cefoxitin and the bioactive material was implemented as follows: a synergistic outcome was observed at an FIC value ≤0.5, an additive outcome was observed at an FIC value >0.5-1.0, an indifferent outcome was equal to the effect observed from the most active component at an FIC value >1.0-2.0, and an antagonistic outcome was observed at FIC values >2.0. The FICs obtained for both MRSA LA and MRSA 81 M show an inverse relation for FIC values with increasing concentration of bioactive metabolites (FIG. 1c).

The FIC values for MRSA 81 M indicate a synergistic combinatory effect for all three CFSM concentrations, whereas the FIC values for MRSA LA only show a synergistic effect at the highest concentration of bioactive metabolites (FIC=0.5; 60 mg/mL). This indicates a strain-specific effect, and the MRSA LA strain could be less sensitive to the bioactive due to the possible presence of agr-deficient mutants or “quorum cheaters” that have been shown to secure fitness benefits over MRSA cells that possess typical agr QS systems^{6,27}. Bioactive-only control wells showed no negative effects on MRSA growth for both strains, indicating that the mechanism of action of the bioactive metabolites is not inherently antimicrobial against MRSA, and that MRSA growth inhibition was only observed when combined with cefoxitin.

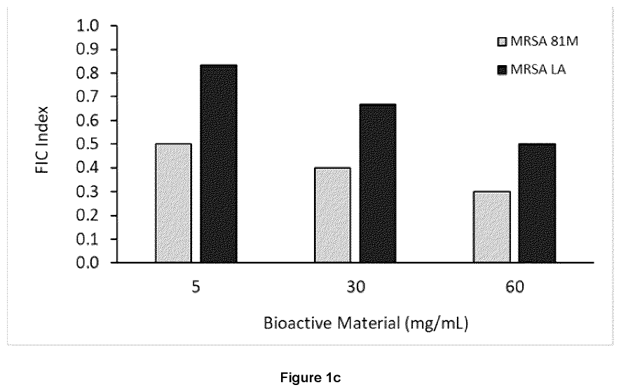

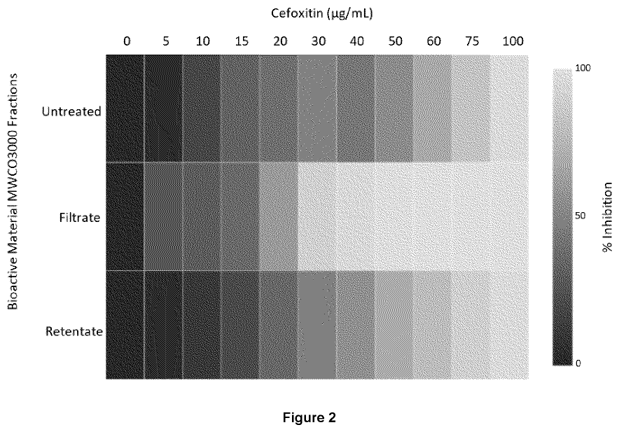

Following the determination of the FIC values, one MRSA strain and one bioactive metabolite concentration (30 mg/mL) was selected. Only MRSA 81 M was investigated as it appeared to have greater sensitivity to the bioactive material and showed synergistic FIC values at all tested concentrations. The bioactive material was split into two fractions using a 3000 MWCO centrifugal filter, with the first fraction containing only the filtrate (<3000 Da) and the second fraction containing only the washed and resuspended retentate (>3000 Da). A checkerboard method was again used to determine the MIC of the MRSA 81M strain using the same combination of cefoxitin concentrations (0-100 μg/mL) with equal starting inoculum (FIG. 2).

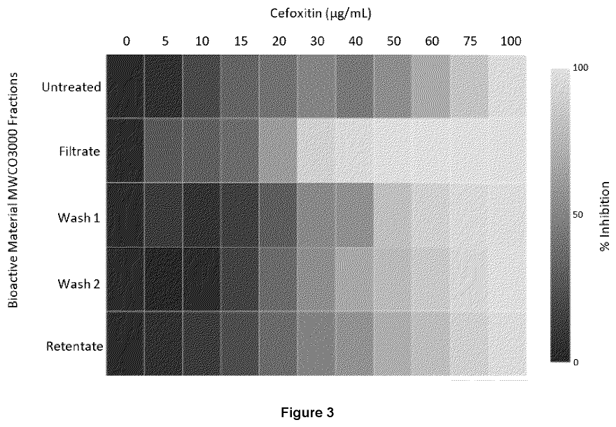

The MIC results of the bioactive fractions clearly show that the filtrate contained the greatest bioactivity and reduced the MIC of cefoxitin; however, some residual activity was detected in the retentate, which resulted in the MIC being slightly less than the untreated control. Moreover, two washes of the retentate that were performed also showed some residual bioactive activity (FIG. 3). These results indicate that the majority of compounds containing bioactivity against the two MRSA clinical strains tested are around 3000 Da or smaller in size.

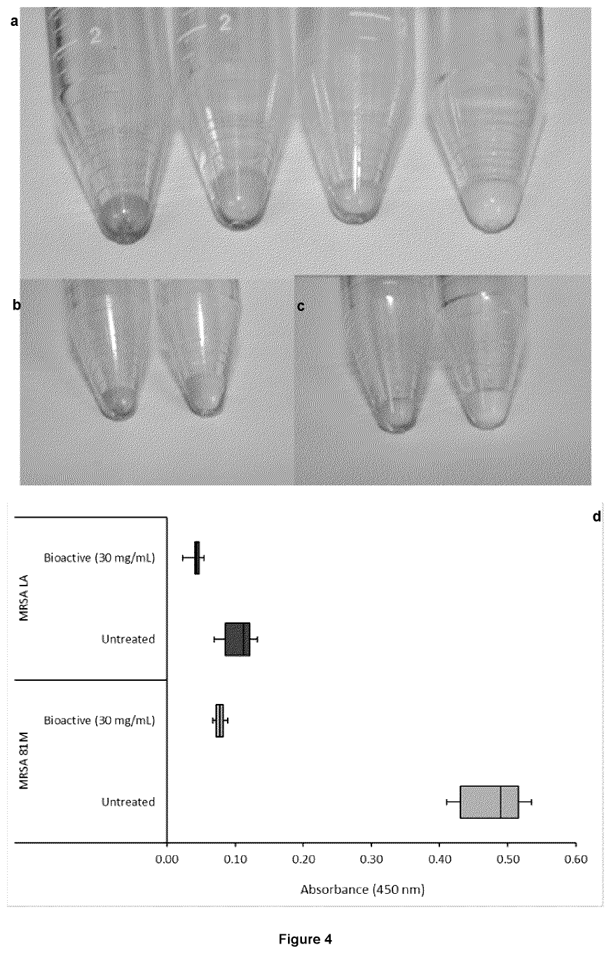

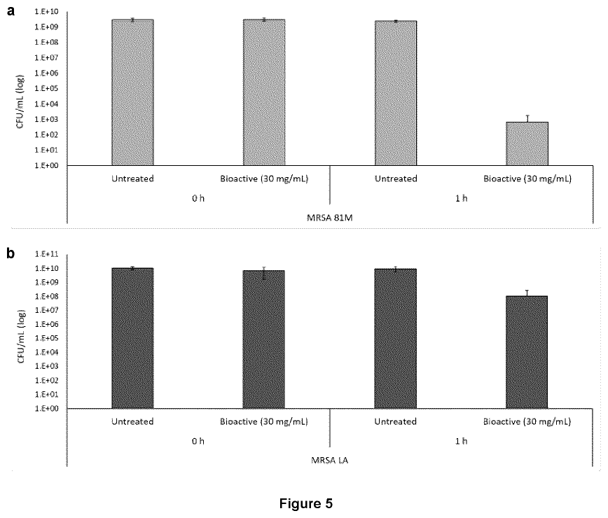

It was observed that following 24 h incubation at 37° C.±1° C. with the bioactive metabolites the MRSA cell pellets became visually lighter and lost the typical yellow-orange coloration characteristic of the majority of MRSA strains. Surface-accumulated compounds called carotenoids contribute to the overall virulence of S. aureus by providing protection against photosensitization, and desiccation, as well as by acting as potent antioxidants that quench toxic reactive oxygen species that are used by immune cells. Following incubation with bioactive metabolites, the orange coloration of the MRSA 81M strain was reduced (FIG. 4a, b). The MRSA LA strain was less impacted as this particular clinical strain exhibited very little carotenoid production (FIG. 4a, c). Although the color difference between treatments was more pronounced in the MRSA 81 M strain, the MRSA LA cells treated with the bioactive metabolites lost their characteristic yellow-orange color and appeared nearly white. Absorbance measurements at 450 nm (A₄₅₀) corroborated the visual observation as there was over a six-fold difference in carotenoid concentration (as measured by absorbance) between treatments of the MRSA 81 M cells, but less than three-fold difference in carotenoid between treatments of the MRSA LA cells (FIG. 4d).

The change in cell pellet color following 24 h incubation with the bioactive metabolites is indicative of probiotic inhibition of MRSA replication and disruption of proper cell wall structuring; carotenoids are also thought to be involved in stabilizing the S. aureus membrane during infection and pathogenesis and maintaining cell rigidity against host defenses^{25,26}. The reduction in characteristic yellow-orange coloring of colloquially-known “Golden Staph” MRSA is likely due to the disruption of QS-related two component systems that are reported to be essential to hydrogen peroxide susceptibility in S. aureus. Moreover, although the cell pellets were nearly identical in total CFU/mL counts between the untreated control and the bioactive-treated MRSA cells, the cell pellets were less dense and formed larger cell pellets, also indicating that the bioactive metabolites affected the synthesis of the cell wall structure of the bioactive-treated MRSA cell. As carotenoid pigments greatly contribute to Staphylococcal fitness, particularly in immune cell evasion, the inhibition of carotenoid synthesis could be a potential target for therapeutic intervention in S. aureus infections²³.

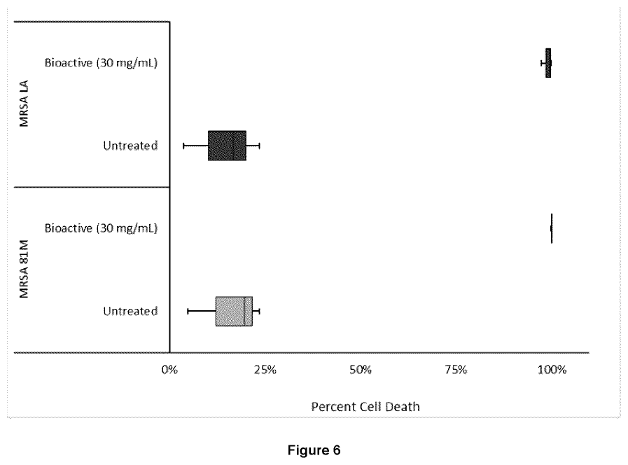

Following the observation of reduced carotenoid synthesis, it was hypothesized that the bioactive metabolites would increase the sensitivity of the MRSA strains to oxidant killing, which is an important mechanism of action of neutrophils against bacterial infections. Hydrogen peroxide was added at 1.5% v/v to untreated and bioactive-treated cultures of each respective MRSA clinical strain and incubated at 37° C.±1° C. for 1 h in PBS solution. Cell counts (CFU/mL) were determined both before and after the 1 h incubation. The bioactive-treated cells showed 99.67% and >99.99% cell death for MRSA LA and 81 M, respectively; as opposed to the untreated MRSA LA and 81 M cells, which exhibited only 16.67% and 19.70% cell death, respectively (FIG. 6). The cell count for bioactive-treated MRSA 81M was nearly seven-fold less than the untreated control, whereas the counts for the bioactive-treated MRSA LA was only two- to three-fold less than the untreated control.

Together, these findings involving the treatment of clinical MRSA strains with postbiotic metabolites supports the notion that certain postbiotic metabolites disrupt typical cell wall synthesis in MRSA by blocking the QS-based regulation of the synthesis of carotenoids that act as crucial antioxidants, resulting in increased sensitivity to oxidant killing. As MRSA LA appeared to have little carotenoid production (compared to MRSA 81M), an alternative theory as to the lower cell death from hydrogen peroxide is that the bioactive metabolites are also able to disrupt the intrinsic component of the agr system responsible for sensing oxidative environments⁸.

In conclusion, we found that the metabolites isolated from the CFSM of Enterococcus faecium aid in increasing the sensitivity of clinical MRSA strains to cefoxitin, a β-lactam antibiotic that is no longer clinically suitable against MRSA infections. As cefoxitin works by impeding the cross-linking of peptidoglycan layers within the bacterial cell wall, the destructive impact of the probiotic bioactives on cell wall synthesis is synergistically beneficial to cefoxitin's mechanism of action. Our data indicate that bioactive metabolites can act as non-antimicrobial adjuvants in conjunction with an antibiotic, and critically, may have the potential to “re-sensitize” MRSA to no longer clinically relevant antibiotics such as cefoxitin. Our results also indicate that these Enterococcus faecium bioactives can impede virulence traits that support MRSA infections; resistance to oxygen species, staphyloxanthin and carotenoid production as well as cell wall synthesis phenotypes mediated by the agr system in S. aureus are also greatly obstructed by these compounds, resulting in increased oxidant cytotoxicity and an altered morphology of clinical MRSA strains. Adjuvant technologies developed from probiotic bacteria that initiate interruption of S. aureus QS systems may potentially be serious contenders in combating MRSA infections.

### REFERENCES

1. Podolski, S. H. The evolving response to antibiotic resistance (1945-2018). Nature Palgrave Communications 4, 1-8 (2018).
2. Turner, N. A. et al. Methicillin-resistant Staphylococcus aureus: an overview of basic and clinical research. Nature Reviews Microbiology, 17, 203-218 (2019).
3. Tyers, M., Wright, G. D. Drug combinations: a strategy to extend the life of antibiotics in the 21st century. Nature Reviews, 17 (3), 141-155 (2019).
4. Holmes, A. H. et al. Understanding the mechanisms and drivers of antimicrobial resistance. The Lancet. 387, 176-187 (2016).
5. Wright, G. D. Antibiotic adjuvants: rescuing antibiotics from resistance. Trends Micb. 24 (11), 862-871 (2016).
6. Kong, K. F., Vuong, C., Otto, M. Staphylococcus quorum sensing in biofilm formation and infection. International Journal of Medical Microbiology, 296 (2-3), 133-139 (2006).
7. Rémy B, Mion S, Plener L, Elias M, Chabrière E and Daudé D. Interference in bacterial quorum sensing: a biopharmaceutical perspective. Front. Pharmacol. 9, 1-17 (2018).
8. Fetzner, S. Quorum quenching enzymes. J Biotech, 201, 2-14 (2015).
9. Otto, M. Quorum-sensing control in Staphylococci—a target for antimicrobial drug therapy? FEMS Micb Letters, 41, 135-141 (2004).
10. Bovbjerg Rasmussen, T. et al. Screening for quorum-sensing inhibitors (QSI) by use of a novel genetic system, the QSI Selector. J. Bacteriology 187 (5), 1799-1814 (2005).
11. Medellin Pena, M. J., and Griffiths, M. W. Effect of molecules secreted by Lactobacillus acidophilus strain La-5 on Escherichia coli O157:H7 colonization. Applied and Envi Microbiology, 75 (4), 1165-1172 (2008).
12. Bayoumi, M. A., and Griffiths, M. W. Probiotics down-regulate genes in Salmonella enterica Serovar Typhimurium pathogenicity islands 1 and 2. J. Food Protection, 73 (3), 452-460 (2010).
13. Bayoumi, M. A., and Griffiths, M. W. In vitro inhibition of expression of virulence genes responsible for colonization and systemic spread of enteric pathogens using Bifidobacterium bifidum secreted molecules. International J. Food Micb, 156 (3), 255-263 (2012).
14. Bondue, P. et al. Cell-free spent media obtained from Bifidobacterium bifidum and Bifidobacterium crudilactis grown in media supplemented with 3′-sialyllactose modulate virulence gene expression in Escherichia coli O157:H7 and Salmonella Typhimurium. Front Micb, 7 (2016).
15. Piewngam, P. et al. Pathogen elimination by probiotic Bacillus via signalling interference. Nature. 562 (7728), 532-537, (2018).
16. Ng, W. L., Bassler, B. L. Bacterial quorum-sensing network architectures. Annu Rev Genet. 43, 197-222 (2009).
17. Yarwood, J. M., and Schlievert, P. M. Quorum sensing in Staphylococcus infections. J. Clinical Investigation, 112 (11), 1620-1625 (2003).
18. Sun, F. et al. Quorum-sensing agr mediates bacterial oxidation response via an intramolecular disulfideredox switch in the response regulator AgrA. Proceedings National Academy of Sciences of the United States of America, 109 (23), 9095-9100 (2012).
19. Oogai, Y., Kawada-Matsuo, M., Komatsuzawa, H. Staphylococcus aureus SrrAB affects susceptibility to hydrogen peroxide and co-existence with Streptococcus sanguinis. Plos One, 11 (7) (2016).
20. Le, K. Y. and Otto, M. Quorum-sensing regulation in staphylococci—an overview. Front. Microbiol. 6, 1174 (2015).
21. Cheung, G. Y. C. et al. Role of the accessory gene regulator agr in community-associated methicillin-resistant Staphylococcus aureus pathogenesis. Infection and Immunity, 79 (5), 1927-1935 (2011).
22. Otto, M. Basis of virulence in community-associated methicillin-resistant Staphylococcus aureus. Annu. Rev. Microbiol. 64, 143-162 (2010).
23. Queck, S. Y. et al. RNAIII-independent target gene control by the agr quorum-sensing system: insight into the evolution of virulence regulation in Staphylococcus aureus. Molecular Cell, 32, 150-158 (2008).
24. Liu, G. Y., et al. Staphylococcus aureus golden pigment impairs neutrophil killing and promotes virulence through its antioxidant activity. Journal of Experimental Medicine, 202 (2), 209-215 (2005).
25. Mishra, N. N. et al. Carotenoid-related alteration of cell membrane fluidity impacts Staphylococcus aureus susceptibility to host defense peptides. Antimicrob. Agents Chemother. 55, 526-531 (2011).
26. Ejim, L. et al. Combinations of antibiotics and nonantibiotic drugs enhance antimicrobial efficacy. Nature Chemical Biology, 7, 348-350 (2011).
27. He, L. et al. Resistance to leukocytes ties benefits of quorum sensing dysfunctionality to biofilm infection. Nature Microbiology Letters, 1-6 (2019).
28. Chamberlain, N. R. et al. Correlation of carotenoid production, decreased membrane fluidity, and resistance to oleic acid killing in Staphylococcus aureus 18Z. Infection and Immunity, 59(12), 4332-4337 (1991).
29. Martineau, F., et al. Correlation between the resistance genotype determined by multiplex PCR assays and the antibiotic susceptibility patterns of Staphylococcus aureus and Staphylococcus epidermidis. Antimicrob Agents Chemother, 44 (2), 231-238 (2000).
30. Clinical and Laboratory Standards Institute. Performance Standards for Antimicrobial Susceptibility Testing; Twenty-Fifth Informational Supplement. CLSI document M100-S25 (ISBN 1-56238-989-0 [Print]; ISBN 1-56238-990-4 [Electronic]). Clinical and Laboratory Standards Institute, 950 West Valley Road, Suite 2500, Wayne, Pa. 19087 USA (2015).
31. Müller, S. et al. Poorly cross-linked peptidoglycan in MRSA due to mecA induction activates the inflammasome and exacerbates immunopathology. Cell Host Microbe, 18, 604-612 (2015).
32. European Committee for Antimicrobial Susceptibility Testing (EUCAST). Terminology relating to methods for the determination of susceptibility of bacteria to antimicrobial agents. EUCAST Definitive Document E.Def 1.2. Clinical Microbiology and Infection, 6 (9) (2000).

### Example 5: Enterococcus faecium Postbiotic Metabolite-Containing CFSM and Cefoxitin Synergistically Reduce Growth of Methicillin-Resistant Staphylococcus Pseudintermidius (MRSP)

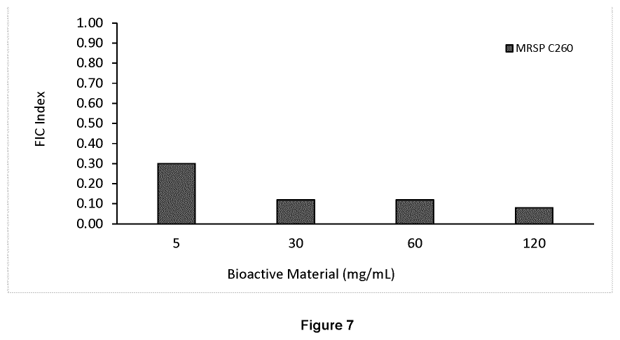

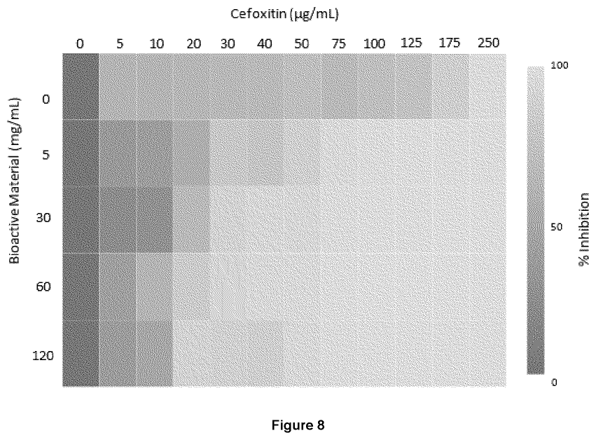

This example was carried out using methods as described above in Example 4. As shown in FIG. 7, cefoxitin and the cell-free supernatant from DSM13241, containing postbiotic metabolites, acted synergistically to treat Staphylococcus pseudintermidius. A heat plot of the MIC percent growth inhibition of MRSP by combination testing of the β-lactam antibiotic cefoxitin and Enterococcus faecium bioactive metabolites is shown in FIG. 8.

The above disclosure generally describes the present invention. Although specific terms have been employed herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

1. A synergistic combination comprising a quorum-sensing inhibitor and/or a postbiotic metabolite and an antibiotic.

2. The synergistic combination of claim 1, wherein the quorum-sensing inhibitor and/or postbiotic metabolite comprises a peptide, small molecule, lipid, sugar, or a combination thereof.

3. The synergistic combination of claim 2, wherein the peptide comprises or consists of one or more of the following amino acid sequences: XX[L or I]PPK, wherein X designates a hydrophobic amino acid; X₁X₂[L or I]PPK, wherein X₁ is selected from N, C, Q, M, S, and T and wherein X₂ is selected from A, I, L, and V; MALPPK; CVLPPK; HLLPLP; LKPTPEGD; YPVEPF; YPPGGP; YPPG; NQPY; LPVPK; ALPK; EVLNCLALPK; LPLP; HLLPLPL; YVPEPF; KYVPEPF; EMPFKPYPVEPF; and variants thereof altered by deletion, substitution or insertion wherein the activity of the molecules is not substantially reduced, including peptides and/or variants thereof with post-translational modifications including glycosylation.

4. The synergistic combination of any one of claims 1 to 3, wherein the quorum-sensing inhibitor and/or postbiotic metabolite is a peptide and comprises or consists of 4, 5, 6, 7, 8, 9, 10, 11, 12, 13, 14, 15, 16, 17, 18, 19, or 20 amino acid residues, such as from 2, 3, 4, 5, 6, 7, 8, or 9 to about 3, 4, 5, 6, 7, 8, 9, or 10 amino residues.

5. The synergistic combination of any one of claims 1 to 4, wherein the quorum-sensing inhibitor and/or postbiotic metabolite is less than about 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, or 500 Da in size, such as less than about 3000, 2000, or 1000 Da in size.

6. The synergistic combination of any one of claims 1 to 5, wherein the quorum-sensing inhibitor and/or postbiotic metabolite is in a probiotic bacterial culture fraction, such as a supernatant.

7. The synergistic combination of claim 6, wherein the probiotic bacterial culture fraction is a cell-free spent medium (CSFM), which is optionally concentrated in liquid or dry form (e.g. by lyophilization and/or spray-drying).

8. The synergistic combination of claim 7, wherein the CSFM has a molecular weight cut-off of about 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, or 500 Da in size, such as about 3000, 2000, or 1000 Da.

9. The synergistic combination of any one of claims 1 to 8, wherein the antibiotic is an aminoglycoside, a bacitracin, a beta-lactam antibiotic, a cephalosporin, a chloramphenicol, a glycopeptide, a macrolides, a lincosamide, a penicillin, a quinolone, a rifampin, a glycopeptide, a tetracycline, a trimethoprim, a sulfonamides, or a combination thereof.

10. The synergistic combination of claim 9, wherein the antibiotic is a β-lactam antibiotic, such as cefoxitin.

11. The synergistic combination of any one of claims 1 to 10, wherein the quorum-sensing inhibitor and/or postbiotic metabolite is derived from the culture medium or supernatant of probiotic bacteria of the genera Aerococcus, Bacillus, Bacteroides, Bifidobacterium, Clostridium, Enterococcus, Fusobacterium, Lactobacillus, Lactococcus, Leuconostoc, Melissococcus, Micrococcus, Pediococcus, Peptostrepococcus, Propionibacterium, Staphylococcus, Streptococcus, Weissella, or combinations thereof.

12. The synergistic combination of claim 11, wherein the quorum-sensing inhibitor and/or postbiotic metabolite is derived from Lactobacillus, such as Lactobacillus acidophilus, such as Lactobacillus acidophilus (DSM13241) and/or wherein the quorum-sensing inhibitor and/or postbiotic metabolite is derived from Enterococcus, such as Enterococcus faecium.

13. The synergistic combination of any one of claims 1 to 12, further comprising a probiotic.

14. The synergistic combination of claim 13, wherein the probiotic is of the genera Aerococcus, Bacillus, Bacteroides, Bifidobacterium, Clostridium, Enterococcus, Fusobacterium, Lactobacillus, Lactococcus, Leuconostoc, Melissococcus, Micrococcus, Pediococcus, Peptostrepococcus, Propionibacterium, Staphylococcus, Streptococcus, Weissella, or combinations thereof.

15. The synergistic combination of claim 14, wherein the probiotic is Lactobacillus, such as Lactobacillus acidophilus, such as Lactobacillus acidophilus (DSM13241) and/or Enterococcus, such as Enterococcus faecium.

16. The synergistic combination of any one of claims 13 to 15, wherein the probiotic is live.

17. The synergistic combination of any one of claims 13 to 16, wherein the probiotic is present in an amount of about 100 million to about 500 million CFU per dose, such as about 200 million CFU per dose.

18. A composition comprising the synergistic combination of any one of claims 1 to 17.

19. A method for:
(a) resensitizing an antibiotic-resistant infection to an antibiotic, the method comprising administering a quorum-sensing inhibitor and/or a postbiotic metabolite to a subject afflicted with the infection;
(b) decreasing resistance of a bacterial infection to hydrogen peroxide cytotoxicity, the method comprising administering a quorum-sensing inhibitor and/or a postbiotic metabolite to a subject afflicted with the infection;
(c) treating and/or preventing diarrhea in a subject, the method comprising administering a quorum-sensing inhibitor and/or a postbiotic metabolite and an antibiotic to the subject;
(d) treating MRSA or MRSP, the method comprising administering a quorum-sensing inhibitor and/or a postbiotic metabolite and an antibiotic to a subject afflicted with MRSA or MRSP;
(e) reducing carotenoid synthesis in bacteria, the method comprising administering a quorum-sensing inhibitor and/or a postbiotic metabolite to the bacteria; and/or
(Ff) sensitizing bacteria to oxidant killing, the method comprising administering a quorum-sensing inhibitor and/or a postbiotic metabolite to the bacteria.

20. The method of claim 19, wherein the antibiotic is an aminoglycoside, a bacitracin, a beta-lactam antibiotic, a cephalosporin, a chloramphenicol, a glycopeptide, a macrolides, a lincosamide, a penicillin, a quinolone, a rifampin, a glycopeptide, a tetracycline, a trimethoprim, a sulfonamides, or a combination thereof.

21. The method of claim 20, wherein the antibiotic is a β-lactam antibiotic, such as cefoxitin.

22. The method of any one of claims 19 to 21, wherein the quorum-sensing inhibitor and/or postbiotic metabolite comprises a peptide, small molecule, lipid, sugar, or a combination thereof.

23. The method of claim 22, wherein the peptide comprises or consists of one or more of the following amino acid sequences: XX[L or I]PPK, wherein X designates a hydrophobic amino acid; X₁X₂[L or I]PPK, wherein X₁ is selected from N, C, Q, M, S, and T and wherein X₂ is selected from A, I, L, and V; MALPPK; CVLPPK; HLLPLP; LKPTPEGD; YPVEPF; YPPGGP; YPPG; NQPY; LPVPK; ALPK; EVLNCLALPK; LPLP; HLLPLPL; YVPEPF; KYVPEPF; EMPFKPYPVEPF; and variants thereof altered by deletion, substitution or insertion wherein the activity of the molecules is not substantially reduced, including peptides and/or variants thereof with post-translational modifications including glycosylation.

24. The method of any one of claims 19 to 23, wherein the quorum-sensing inhibitor and/or postbiotic metabolite is a peptide and comprises or consists of 4, 5, 6, 7, 8, 9, 10, 11, 12, 13, 14, 15, 16, 17, 18, 19, or 20 amino acid residues, such as from 2, 3, 4, 5, 6, 7, 8, or 9 to about 3, 4, 5, 6, 7, 8, 9, or 10 amino residues.

25. The method of any one of claims 19 to 24, wherein the quorum-sensing inhibitor and/or postbiotic metabolite is less than about 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, or 500 Da in size, such as less than about 3000, 2000, or 1000 Da in size.

26. The method of any one of claims 19 to 25, wherein the quorum-sensing inhibitor and/or postbiotic metabolite is in a probiotic bacterial culture fraction, such as a supernatant.

27. The method of claim 26, wherein the probiotic bacterial culture fraction is a cell-free spent medium (CSFM), which is optionally concentrated in liquid or dry form (e.g. by lyophilization and/or spray-drying).

28. The method of claim 27, wherein the CSFM has a molecular weight cut-off of about 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, or 500 Da in size, such as about 3000, 2000, or 1000 Da.

29. The method of any one of claims 19 to 28, wherein the quorum-sensing inhibitor and/or postbiotic metabolite is derived from the culture medium or supernatant of probiotic bacteria of the genera Aerococcus, Bacillus, Bacteroides, Bifidobacterium, Clostridium, Enterococcus, Fusobacterium, Lactobacillus, Lactococcus, Leuconostoc, Melissococcus, Micrococcus, Pediococcus, Peptostrepococcus, Propionibacterium, Staphylococcus, Streptococcus, Weissella, or combinations thereof.

30. The method of claim 29, wherein the quorum-sensing inhibitor and/or postbiotic metabolite is derived from Lactobacillus, such as Lactobacillus acidophilus, such as Lactobacillus acidophilus (DSM13241) and/or wherein the quorum-sensing inhibitor and/or postbiotic metabolite is derived from Enterococcus, such as Enterococcus faecium.

31. The method of any one of claims 19 to 30, further comprising administering a probiotic to the subject.

32. The method of claim 31, wherein the probiotic is of the genera Aerococcus, Bacillus, Bacteroides, Bifidobacterium, Clostridium, Enterococcus, Fusobacterium, Lactobacillus, Lactococcus, Leuconostoc, Melissococcus, Micrococcus, Pediococcus, Peptostrepococcus, Propionibacterium, Staphylococcus, Streptococcus, Weissella, or combinations thereof.

33. The method of claim 32, wherein the probiotic is Lactobacillus, such as Lactobacillus acidophilus, such as Lactobacillus acidophilus (DSM13241) and/or wherein the quorum-sensing inhibitor and/or postbiotic metabolite is derived from Enterococcus, such as Enterococcus faecium.

34. The method of any one of claims 31 to 33, wherein the probiotic is live.

35. The method of any one of claims 31 to 34, wherein the probiotic is present in an amount of about 100 million to about 500 million CFU per dose, such as about 200 million CFU per dose.

36. The method of any one of claims 19 to 35, wherein the subject is a pet, such as a dog.

37. The method of any one of claims 19 to 35, wherein the subject is a farm animal, such as swine or poultry.

38. The method of any one of claims 19 to 35, wherein the subject is a human.

39. The method of any one of claims 19 to 38, wherein the quorum-sensing inhibitor and/or postbiotic metabolite and the antibiotic act synergistically.

40. A tablet or capsule comprising the synergistic combination of any one of claims 1 to 17 or the composition of claim 18.

41. Use of the synergistic combination of any one of claims 1 to 17 or the composition of claim 18 in the method of any one of claims 19 to 39.

42. The synergistic combination of any one of claims 1 to 17 or the composition of claim 18 for use in the method of any one of claims 19 to 39.
